(19) United States
(12) Patent Application Publication
(10) Pub. No.: US 2022/0001019 A1
(43) Pub. Date: Jan. 6, 2022

(54) RANDOM CONJUGATE MOLECULE AND METHODS OF MAKING AND USING THE SAME

(71) Applicant: The Regents of the University of California, Oakland, CA (US)

(72) Inventors: B. Chia Soo, Los Angeles, CA (US); Kang Ting, Los Angeles, CA (US); Benjamin M. Wu, San Marino, CA (US)

(21) Appl. No.: 16/839,717

(22) Filed: Apr. 3, 2020

Related U.S. Application Data
(63) Continuation of application No. 15/068,412, filed on Mar. 11, 2016, now abandoned.
(60) Provisional application No. 62/133,226, filed on Mar. 13, 2015.

(51) Int. Cl. A61K 47/64; A61K 38/19; A61K 47/60; A61K 9/00

(52) U.S. Cl. CPC A61K 47/64; A61K 9/0019; A61K 47/60; A61K 38/19

(57) ABSTRACT

The present invention discloses a random conjugate molecule (CMN), comprising a formula of CG-X-N and methods of making and using the same. The present invention also discloses a composition comprising the CMN and methods of making and using the composition.

### CROSS-REFERENCE TO RELATED APPLICATIONS

This is a continuation of U.S. application Ser. No. 15/068,412, filed Mar. 11, 2016, which claims the benefit of U.S. provisional application No. 62/133,226, filed on Mar. 13, 2015, the teaching of which is incorporated herein by reference in its entirety.

### STATEMENT OF GOVERNMENTAL SUPPORT

This invention was made with Government support under Grant No. NIH R01AR066782 awarded by the National Institutes of Health (NIH). The Government has certain rights in the invention.

### FIELD OF THE INVENTION

The present invention generally relates a random conjugate molecule and methods of making and using the same and compositions thereof and methods of making and using the composition.

### BACKGROUND OF THE INVENTION

With an aging population, the biomedical burden of osteoporosis is significantly escalating, with no novel therapeutic to address systemic bone loss. NELL-1 is an osteoinductive factor recently discovered to induce bone formation and reverse osteoporotic bone loss when administered intravenously. However, unmodified NELL-1 requires an impractical 48 hr injection frequency and thus limits NELL-1's translation into a clinical setting.

Therefore, there is a continuing need for agents for treating or ameliorating osteoporosis. The embodiments below address the above described problems and needs.

### SUMMARY OF THE INVENTION

In one aspect of the present invention, it is provided a random conjugate molecule (CMN), comprising a formula of CG-X-N,

wherein:
CG is at least one chemical group,
X is a linking group, and
N is NELL-1 protein or peptide.

In some embodiments of the invention molecule, optionally in combination with any or all of the various embodiments disclosed herein, the at least one chemical group is an alkyl group, an aryl group, an acyl group, a leaving group, a polymer, or a peptide, or a combination thereof.

In some embodiments of the invention molecule, optionally in combination with any or all of the various embodiments disclosed herein, the chemical group is poly(ethylene glycol) (PEG), poly(ethylene oxide) (PEO), poly(propylene glycol) (PPG), or poly(propylene oxide) (PPO).

In some embodiments of the invention molecule, optionally in combination with any or all of the various embodiments disclosed herein, the chemical group is selected from heparin sulfate, glycopolymers, zwitterionic polymers, hyperbranched polymers, polymers containing unnatural amino acids, linkers to lysine or cysteine, or peptide sequences that modify NN or CMN interactions with ECM, target cells, immune cells, and hepatocytes.

In some embodiments of the invention molecule, optionally in combination with any or all of the various embodiments disclosed herein, the linking group comprises a responsive linker that degrades on demand to external stimuli.

In some embodiments of the invention molecule, optionally in combination with any or all of the various embodiments disclosed herein, the linking group provides a linking reaction that is azide-alkyne, azide-BMCO, or Tetrazine-TCO type reactions.

In some embodiments of the invention molecule, optionally in combination with any or all of the various embodiments disclosed herein, the linking group comprises a linker to lysine or cysteine.

In some embodiments of the invention molecule, optionally in combination with any or all of the various embodiments disclosed herein, the linking group comprises a natural enzyme.

In some embodiments of the invention molecule, optionally in combination with any or all of the various embodiments disclosed herein, the at least one chemical group imparts at least one desirable property to the NELL-1 protein such that the CMN is significantly improved in the at least one desirable property relative to a naked NELL-1 protein without chemical modification (NN).

In some embodiments of the invention molecule, optionally in combination with any or all of the various embodiments of the present invention, the at least one desirable property is selected from the group consisting of blood circulation life, shelf-life, hydrophobicity or hydrophilicity, biological activity, bioavailability, cytotoxicity, non-immunogenicity, or conformational properties, etc.

In another aspect of the present invention, it is provided a composition comprising a random conjugate molecule (CMN) of a formula of CG-X-N,

wherein:
CG is at least one chemical group,
X is a linking group, and
N is NELL-1 protein or peptide.

In some embodiments of the invention composition, optionally in combination with any or all of the various embodiments disclosed herein, the at least one chemical group is an alkyl group, an aryl group, an acyl group, a leaving group, a polymer, or a peptide, or a combination thereof.

In some embodiments of the invention composition, optionally in combination with any or all of the various embodiments disclosed herein, the chemical group is poly(ethylene glycol) (PEG), poly(ethylene oxide) (PEO), poly(propylene glycol) (PPG), or poly(propylene oxide) (PPO).

In some embodiments of the invention composition, optionally in combination with any or all of the various embodiments disclosed herein, the chemical group is selected from heparin sulfate, glycopolymers, zwitterionic polymers, hyperbranched polymers, polymers containing unnatural amino acids, linkers to lysine or cysteine, or peptide sequences that modify NN or CMN interactions with ECM, target cells, immune cells, and hepatocytes.

In some embodiments of the invention composition, optionally in combination with any or all of the various embodiments disclosed herein, the linking group comprises a responsive linker that degrades on demand to external stimuli.

In some embodiments of the invention composition, optionally in combination with any or all of the various embodiments disclosed herein, the linking group provides a linking reaction that is azide-alkyne, azide-BMCO, or Tetrazine-TCO type reactions.

In some embodiments of the invention composition, optionally in combination with any or all of the various embodiments disclosed herein, the linking group comprises a linker to lysine or cysteine.

In some embodiments of the invention composition, optionally in combination with any or all of the various embodiments disclosed herein, the linking group comprises a natural enzyme.

In some embodiments of the invention composition, optionally in combination with any or all of the various embodiments disclosed herein, the at least one chemical group imparts at least one desirable property to the NELL-1 protein such that the CMN is significantly improved in the at least one desirable property relative to a naked NELL-1 protein without chemical modification (NN).

In some embodiments of the invention composition, optionally in combination with any or all of the various embodiments disclosed herein, the composition further comprises a pharmaceutically acceptable carrier.

In some embodiments of the invention composition, optionally in combination with any or all of the various embodiments disclosed herein, the composition is a formulation for systemic or local delivery.

In some embodiments of the invention composition, optionally in combination with any or all of the various embodiments of the present invention, the at least one desirable property is selected from the group consisting of blood circulation life, shelf-life, hydrophobicity or hydrophilicity, biological activity, bioavailability, cytotoxicity, non-immunogenicity, or conformational properties, etc.

In a further aspect of the present invention, it is provided a method of preparing a random conjugate molecule (CMN) of a formula of CG-X-N, comprising:

a) providing a NELL-1 protein or peptide (N),

b) providing a chemical compound comprising a chemical group (CG),

c) providing a linking group that comprises a linker (X), and

d) bringing the CG into contact with the NELL-1 protein or peptide to cause conjugation between the chemical group and the NELL-1 protein or peptide to occur to form the CMN.

In some embodiments of the invention method, optionally in combination with any or all of the various embodiments disclosed herein, the at least one chemical group is an alkyl group, an aryl group, an acyl group, a leaving group, a polymer, or a peptide, or a combination thereof.

In some embodiments of the invention method, optionally in combination with any or all of the various embodiments disclosed herein, the chemical group is poly(ethylene glycol) (PEG), poly(ethylene oxide) (PEO), poly(propylene glycol) (PPG), or poly(propylene oxide) (PPO).

In some embodiments of the invention method, optionally in combination with any or all of the various embodiments disclosed herein, the chemical group is selected from heparin sulfate, glycopolymers, zwitterionic polymers, hyperbranched polymers, polymers containing unnatural amino acids, linkers to lysine or cysteine, or peptide sequences that modify NN or CMN interactions with ECM, target cells, immune cells, and hepatocytes.

In some embodiments of the invention method, optionally in combination with any or all of the various embodiments disclosed herein, the linking group comprises a responsive linker that degrades on demand to external stimuli.

In some embodiments of the invention method, optionally in combination with any or all of the various embodiments disclosed herein, the linking group provides a linking reaction that is azide-alkyne, azide-BMCO, or Tetrazine-TCO type reactions.

In some embodiments of the invention method, optionally in combination with any or all of the various embodiments disclosed herein, the linking group comprises a linker to lysine or cysteine.

In some embodiments of the invention method, optionally in combination with any or all of the various embodiments disclosed herein, the linking group comprises a natural enzyme.

In some embodiments of the invention method, optionally in combination with any or all of the various embodiments disclosed herein, the at least one chemical group imparts at least one desirable property to the NELL-1 protein such that the CMN is significantly improved in the at least one desirable property relative to a naked NELL-1 protein without chemical modification (NN).

In some embodiments of the invention method, optionally in combination with any or all of the various embodiments of the present invention, the at least one desirable property is selected from the group consisting of blood circulation life, shelf-life, hydrophobicity or hydrophilicity, biological activity, bioavailability, cytotoxicity, non-immunogenicity, or conformational properties, etc.

In a further aspect of the present invention, it is provided a method of forming a composition, comprising:

providing an amount of a random conjugate molecule (CMN) of a formula of CG-X-N, and

forming the composition,

wherein:
CG is at least one chemical group,
X is a linking group, and
N is NELL-1 protein or peptide.

In some embodiments of the invention method, optionally in combination with any or all of the various embodiments disclosed herein, the at least one chemical group is an alkyl group, an aryl group, an acyl group, a leaving group, a polymer, or a peptide, or a combination thereof.

In some embodiments of the invention method, optionally in combination with any or all of the various embodiments disclosed herein, the chemical group is poly(ethylene glycol) (PEG), poly(ethylene oxide) (PEO), poly(propylene glycol) (PPG), or poly(propylene oxide) (PPO).

In some embodiments of the invention method, optionally in combination with any or all of the various embodiments disclosed herein, the chemical group is selected from heparin sulfate, glycopolymers, zwitterionic polymers, hyperbranched polymers, polymers containing unnatural amino acids, linkers to lysine or cysteine, or peptide sequences that modify NN or CMN interactions with ECM, target cells, immune cells, and hepatocytes.

In some embodiments of the invention method, optionally in combination with any or all of the various embodiments disclosed herein, the linking group comprises a responsive linker that degrades on demand to external stimuli.

In some embodiments of the invention method, optionally in combination with any or all of the various embodiments disclosed herein, the linking group provides a linking reaction that is azide-alkyne, azide-BMCO, or Tetrazine-TCO type reactions.

In some embodiments of the invention method, optionally in combination with any or all of the various embodiments disclosed herein, the linking group comprises a linker to lysine or cysteine.

In some embodiments of the invention method, optionally in combination with any or all of the various embodiments disclosed herein, the linking group comprises a natural enzyme.

In some embodiments of the invention method, optionally in combination with any or all of the various embodiments disclosed herein, the at least one chemical group imparts at least one desirable property to the NELL-1 protein such that the CMN is significantly improved in the at least one desirable property relative to a naked NELL-1 protein without chemical modification (NN).

In some embodiments of the invention method, optionally in combination with any or all of the various embodiments disclosed herein, the composition further comprises a pharmaceutically acceptable carrier.

In some embodiments of the invention method, optionally in combination with any or all of the various embodiments disclosed herein, the composition is a formulation for systemic or local delivery.

In some embodiments of the invention method, optionally in combination with any or all of the various embodiments of the present invention, the at least one desirable property is selected from the group consisting of blood circulation life, shelf-life, hydrophobicity or hydrophilicity, biological activity, bioavailability, cytotoxicity, non-immunogenicity, or conformational properties, etc.

In still a further aspect of the present invention, it is provided method of treating or ameliorating a condition in a subject, comprising administering to the subject a random conjugate molecule (CMN) of a formula of CG-X-N,

wherein:
CG is at least one chemical group,
X is a linking group, and
N is NELL-1 protein or peptide.

In some embodiments of the invention method, optionally in combination with any or all of the various embodiments disclosed herein, the at least one chemical group is an alkyl group, an aryl group, an acyl group, a leaving group, a polymer, or a peptide, or a combination thereof.

In some embodiments of the invention molecule, optionally in combination with any or all of the various embodiments disclosed herein, the chemical group is poly(ethylene glycol) (PEG), poly(ethylene oxide) (PEO), poly(propylene glycol) (PPG), or poly(propylene oxide) (PPO).

In some embodiments of the invention method, optionally in combination with any or all of the various embodiments disclosed herein, the chemical group is selected from heparin sulfate, glycopolymers, zwitterionic polymers, hyperbranched polymers, polymers containing unnatural amino acids, linkers to lysine or cysteine, or peptide sequences that modify NN or CMN interactions with ECM, target cells, immune cells, and hepatocytes.

In some embodiments of the invention method, optionally in combination with any or all of the various embodiments disclosed herein, the linking group comprises a responsive linker that degrades on demand to external stimuli.

In some embodiments of the invention method, optionally in combination with any or all of the various embodiments disclosed herein, the linking group provides a linking reaction that is azide-alkyne, azide-BMCO, or Tetrazine-TCO type reactions.

In some embodiments of the invention method, optionally in combination with any or all of the various embodiments disclosed herein, the linking group comprises a linker to lysine or cysteine.

In some embodiments of the invention method, optionally in combination with any or all of the various embodiments disclosed herein, the linking group comprises a natural enzyme.

In some embodiments of the invention method, optionally in combination with any or all of the various embodiments disclosed herein, the at least one chemical group imparts at least one desirable property to the NELL-1 protein such that the CMN is significantly improved in the at least one desirable property relative to a naked NELL-1 protein without chemical modification (NN).

In some embodiments of the invention method, optionally in combination with any or all of the various embodiments of the present invention, the at least one desirable property is selected from the group consisting of blood circulation life, shelf-life, hydrophobicity or hydrophilicity, biological activity, bioavailability, cytotoxicity, non-immunogenicity, or conformational properties, etc.

In some embodiments of the invention method, optionally in combination with any or all of the various embodiments of the present invention, the CMN is included in a composition that comprises the CMN and a pharmaceutically acceptable carrier.

In some embodiments of the invention method, optionally in combination with any or all of the various embodiments of the present invention, the composition is a formulation for local or systemic delivery.

In some embodiments of the invention method, optionally in combination with any or all of the various embodiments of the present invention, the bone condition is osteoporosis.

In some embodiments of the invention method, optionally in combination with any or all of the various embodiments of the present invention, the bone condition is osteoporosis.

In some embodiments of the invention method, optionally in combination with any or all of the various embodiments of the present invention, the bone condition is bone fracture or intervertebral disc disease or injury.

In some embodiments of the invention method, optionally in combination with any or all of the various embodiments of the present invention, wherein administering comprises local or systemic administration.

In some embodiments of the invention method, optionally in combination with any or all of the various embodiments of the present invention, the subject is a human being.

### BRIEF DESCRIPTION OF THE DRAWINGS

FIG. 1a illustrate a PEG/NELL-1 conjugation mechanism. FIG. 1b shows the schematic structures of the three PEGylated NELL-1 with different PEG molecules. FIG. 1c shows the elution curves of the three NELL-PEG conjugates.

FIG. 2a is a plot shown the PEGylation degree of the NELL-PEG that was determined by the fluorescamine method as described above. FIG. 2b shows the amount of the residual amine group and PEG modification degree of each NELL-PEG that were calculated based on the slopes of the linear regression analysis.

FIG. 3a shows the thermal stability of the PEGylated NELL-1 that was investigated by the thermal shift assay; FIG. 3b shows the Tₘ of the proteins that were analyzed by a Boltzmann model and the corresponding thermal shift amount (ΔTₘ=Tₘ−T₀).

FIG. 4 shows the effect of PEG type and concentration on NELL-PEG cytotoxicity that was investigated in MC3T3 cell line by Alamar blue assay.

FIG. 5a shows the relative bioactivity of the NELL-PEG; FIG. 5b shows the ARS in the wells that was next extracted from the hPSC cell monolayer by acetic acid for bioactivity quantification. The bioactivity of the three kinds of NELL-PEG was significantly higher than the NELL-1 free control group (*p<0.05); FIG. 5c shows the relative activity of different PEGylated NELL-1 protein compared to naked NELL-1; FIG. 5d depicts the images of the whole wells containing stained calcium mineralization, which showed that all the protein groups (naked NELL-1, NELL-PEG-5k, NELL-PEG-20k, and NELL-PEG-40k) significantly increased the matrix mineralization compared to the negative control group (NELL-1 free).

FIG. 6 shows the remaining amount (%) of NELL-1 and NELL-PEG in mice at different time points after a single intravenous injection.

FIG. 7 shows mean serum concentration-time curves of naked NELL-1 and NELL-PEG-5k following an intravenous injection in mice. A dose of 1.25 mg/kg NELL-PEG tagged with FITC was injected into 6 mice by the tail vein, and blood samples were collected at a series of time points (0.5, 1, 4, 8, 12, 24, and 36 h). The concentration of PEGylated NELL-1 was then analyzed by monitoring the fluorescence intensity of FITC using a plate reader. The study suggests that the PEGylated NELL-1 has a longer circulation time in mice compared to naked NELL-1.

FIGS. 8A-B show Biodistribution of NELL-PEG-5k and NELL-1 labeled with VivoTag 680XL measured by IVIS Lumina II. CD-1 mice (n=3 per group) were administered intravenously with VivoTag-NELL-PEG-5k, VivoTag-NELL, or a saline control. Organs were imaged ex vivo at 48 hours post injection. (A) Ex vivo fluorescence images of the dissected organs were obtained using the IVIS imaging system. The gradient bar corresponds to the fluorescence intensity. (B) Various organs exhibited different uptakes of VivoTag-NELL-PEG-5k, VivoTag-NELL, and the saline control at 48 hours post tail vein injection. Ex vivo biodistribution confirmed that bones (calvaria, femurs, tibiae, and vertebrae) exhibited a greater retention of PEGylated NELL-1 than naked NELL-1 (*p<0.05).

FIGS. 9A-B show dual-energy X-ray absorptiometry (DXA) taken weekly to monitor bone mineral density (BMD) changes in femur and lumbar vertebrae. (A) (Left) Femoral region-of-interest is shown as red box at distal metaphysis. (Right) At 4 weeks post treatment, femurs treated with q4d and q7d NELL-PEG showed a gradual and significant increase in BMD compared to their respective pre-treatment values. Both NELL-PEG groups also demonstrated statistically significant increases in femoral BMD compared to the PBS/PEG control, but with no considerable difference between each other (##p<0.01 for q4d NELL-PEG, **p<0.01 for q7d NELL-PEG). (B) (Left) Lumbar region-of-interest is shown as red box at L6 vertebral body. (Right) Lumbar vertebrae in both NELL-PEG groups exhibited increasing BMD relative to the baseline throughout the experiment, with greater BMD increments observed in the q7d NELL-PEG group than in the q4d NELL-PEG group. At week 4, lumbar vertebrae treated with q7d NELL-PEG showed significantly higher BMD increases than the vehicle group (*p<0.05 for q7d NELL-PEG). No significant difference in lumbar BMD was observed between the two experimental groups at week 4.

FIGS. 10A-B show Live-[18F] microPET/CT overlay imaging to assess bone metabolic rate. (A) At 4 weeks of treatment, NELL-PEG (q7d) group exhibited an overall greater concentration of [18F]ion, particularly near growth plate areas in the proximal and distal femurs, proximal tibias, proximal humeri and in the vertebral bodies compared to control. Please note that the color intensity in the thoracic vertebrae is due to the overlap of the spinal curvature. (B) Quantitative assessment of [18F] uptake at the distal femur and the terminal lumbar vertebra (L6) was validated by statistical analysis of normalized mean values (% ID/cc). The distal femur showed a statistically significant percent increase of [18F] uptake at week 4 compared to week 0 (B;*p<0.05), and the L6 showed a mean increase in the NELL-PEG group compared to control (data not shown).

FIGS. 11A-F show Post-mortem microCT results at 4 weeks post treatment. (A-E) Femurs treated with q4d and q7d NELL-PEG exhibited considerable increases in (A) the trabecular BMD, (B) the bone volume density (BV/TY), and various structural parameters including (C) trabecular thickness (Tb.Th), (D) trabecular number (Tb.N), and (E) trabecular spacing (Tb.Sp) compared to the PBS/PEG control. Both NELL-PEG groups demonstrated statistically significant BV/TY increases compared to the control, but with no significant difference between each other. Notably, the q7d NELL-PEG group exhibited significant improvement in all other trabecular values (BMD, Tb.Th, and Tb.N) compared to the control group (*p<0.05, **p<0.01). (F) 3D reconstructions of representative femurs in the volume of analysis (above; 3 mm in height) and near the growth plate (bottom).

FIGS. 12A-B show colony-forming unit-fibroblast (CFU-F) assay results from NELL-PEG (q7d) and PBS/PEG control groups. (A) CFU-F-derived colonies were stained using Giemsa Stain Solution at 4 weeks post treatment. (B) BMSC content was quantified by counting colonies microscopically. The q7d NELL-PEG group exhibited a significant increase in BMSC content compared to the control group (*p<0.05).

FIGS. 13A-F show histological and immunohistochemical staining of NELL-PEG (q7d) and PBS/PEG control groups. (A) H&E and (B) Trichrome staining exhibited greater trabecular bone formation at the distal femoral metaphysis in the q7d NELL-PEG group compared to control. (C) Osteocalcin (OCN) immunostaining exhibited a greater number of OCN positive cells with intense staining in the NELL-PEG group, and (D) TRAP staining exhibited a reduction in TRAP positive cells in the NELL-PEG treated femurs compared to control. (E, F) Quantification of OCN+ cells per trabecular bone perimeter (mm-1)(E) and TRAP+ cells per trabecular bone perimeter (mm-1)(F) were averaged from six random fields per sample. OCN was significantly increased and TRAP significantly decreased in NELL-PEG (q7d) group compared to PBS/PEG control by 4 weeks of treatment (*p<0.01).

FIGS. 14A-B show OVX induction of osteoporosis was confirmed by an 11% mean reduction in BMD, at which time intravenous NELL-1 therapy was instituted. After 4 weeks of systemic administration, NELL-1 treatment resulted in a significant increase in BMD in both OVX and Sham-operated animals; FIG. 14C shows post-mortem microCT examination of distal femurs and lumbar spines confirmed that animals treated with NELL-1 possessed increased bone density and bone volume; FIG. 14D shows that when attaching 40K Da of branched PEG to NELL-1, half-life was 29.4 hours.

FIGS. 15A-B show that osteoporosis induction was confirmed by an 11% mean reduction in BMD, upon which intravenous NELL-1 therapy was instituted. IV NELL-1 treatment resulted in a gradual increase in BMD in both Sham- and OVX-treated animals; FIG. 15C shows microCT analysis confirmed a significant increase in all parameters of interest, including BMD, bone volume, and trabecular thickness, observed in both non-osteoporotic and osteoporotic animals; FIG. 15D shows that increased osteoblasts per bone perimeter (B. Pm) were observed with NELL-1 treatment, as confirmed by increased immunostaining for OCN and OPN; FIG. 15E shows the effects of PEGylation on NELL-1 half-life and bioactivity.

FIG. 16 shows the effects of PEGylation on NELL-1 half-life and bioactivity. PEGylation with Linear 5K Da, Linear 20K, and Branched 40K was observed to increase the half-life of NELL-1 from 5.5 hours to 16.1, 31.3, and 29.4 hours, respectively.

FIGS. 17A-B show that at 4 weeks of treatment, bone volume of the fracture site in the PEG-NELL-1 group was 22% greater compared with control across three different threshold values; there were no significant differences in trabecular density.

FIG. 18 shows that microPET demonstrated 34% more uptake of F-18 in the PEG-NELL-1 group compared with control.

FIGS. 19A-F shows on DXA, the PEG-NELL-1 group demonstrated significantly higher BMD than control in the mid femur, distal femur, proximal humerus, and distal humerus.

FIG. 20 shows four schema depicting exemplary chemical modifications of NELL-1 protein. Chemical modification on NELL-1 protein can be achieved through random conjugation of a modifying group to the NELL-1 protein. Schema (1)-(4) illustrate a few exemplary conjugation reactions to prepare and make CMN (chemically modified NELL-1) by so-called click chemistry. See also, Craig S. McKayl and M. G. Finn, “Click Chemistry in Complex Mixtures: Bioorthogonal Bioconjugation” in Chemistry & Biology 21, Sep. 18, 2014, the teaching of which is incorporated herein in its entirety by reference.

FIG. 21 shows a table of pharmacokinetic parameters of NELL-1 and NELL-PEG-5k in mice. The parameters were calculated based on the protein serum concentration at different time-points after injection. NELL-PEG-5k exhibited significant improvements in the elimination half-life time and maximum concentration in blood.

### DETAILED DESCRIPTION OF THE INVENTION

### Definitions

The term “therapeutically effective amount”, as used herein, is an amount of an agent that is sufficient to produce a statistically significant, measurable change of a condition in repaired tissue using the agent disclosed herein as compared with the condition in the repaired tissue without using the agent. Such effective amounts can be gauged in clinical trials as well as animal studies. Such a statistically significant, measurable, and positive change of a condition in repaired tissue using the agent disclosed herein as compared with the condition in the repaired tissue without using the agent is referred to as being an “improved condition”.

As used herein, the term “significantly” or “significant” shall mean statistically significant.

Whenever referred to, the term “chemical group” refers to molecular or polymeric chemical or biochemical compound, which can be natural or synthetic. The chemical compound can include any of the groups disclosed herein above or below.

Whenever referred to, the term “alkyl” whenever used refers to a monovalent alkane (hydrocarbon) derived radical containing from 1 to 10 carbon atoms unless otherwise defined. It may be straight, branched or cyclic. Preferred alkyl groups include methyl, ethyl, propyl, isopropyl, butyl, t-butyl, cyclopentyl and cyclohexyl. When substituted, alkyl groups may be substituted with up to four substituent groups, selected from Rd and Ri, as defined, at any available point of attachment When the alkyl group is said to be substituted with an alkyl group, this is used interchangeably with “branched alkyl group”.

Whenever referred to, cycloalkyl is a specie of alkyl containing from 3 to 15 carbon atoms, without alternating or resonating double bonds between carbon atoms. It may contain from 1 to 4 rings which are fused.

Whenever referred to, the term “alkenyl” refers to a hydrocarbon radical straight, branched or cyclic containing from 2 to 10 carbon atoms and at least one carbon to carbon double bond. Preferred alkenyl groups include ethenyl, propenyl, butenyl and cyclohexenyl.

Whenever referred to, the term “alkynyl” refers to a hydrocarbon radical straight or branched, containing from 2 to 10 carbon atoms and at least one carbon to carbon triple bond. Preferred alkynyl groups include ethynyl, propynyl and butynyl.

Whenever referred to, aryl refers to aromatic rings e.g., phenyl, substituted phenyl and the like, as well as rings which are fused, e.g., naphthyl, phenanthrenyl and the like. An aryl group thus contains at least one ring having at least 6 atoms, with up to five such rings being present, containing up to 22 atoms therein, with alternating (resonating) double bonds between adjacent carbon atoms or suitable heteroatoms. The preferred aryl groups are phenyl, naphthyl and phenanthrenyl. Aryl groups may likewise be substituted as defined. Preferred substituted aryls include phenyl and napbthyl.

Whenever referred to, the term “heteroaryl” refers to a monocyclic aromatic group having 5 or 6 ring atoms, or a bicyclic aromatic group having 8 to 10 atoms, containing at least one heteroatom, O, S or N, in which a carbon or nitrogen atom is the point of attachment, and in which one or two additional carbon atoms is optionally replaced by a heteroatom selected from O or S, and in which from 1 to 3 additional carbon atoms are optionally replaced by nitrogen heteroatoms, said heteroaryl group being optionally substituted as described herein. Examples of this type are pyrrole, pyridine, oxazole, thiazole and oxazine. Additional nitrogen atoms may be present together with the first nitrogen and oxygen or sulfur, giving, e.g., thiadiazole.

Whenever referred to, teteroarylium refers to heteroaryl groups bearing a quaternary nitrogen atom and thus a positive charge.

When a charge is shown on a particular nitrogen atom in a ring which contains one or more additional nitrogen atoms, it is understood that the charge may reside on a different nitrogen atom in the ring by virtue of charge resonance that occurs.

Whenever referred to, the term “heterocycloalkyl” refers to a cycloalkyl group (nonaromatic) in which one of the carbon atoms in the ring is replaced by a heteroatom selected from O, S or N, and in which up to three additional carbon atoms may be replaced by hetero atoms.

Whenever referred to, the terms “quaternary nitrogen” and “positive charge” refer to tetravalent, positively charged nitrogen atoms including, e.g., the positively charged nitrogen in a tetraalkylammonium group (e.g. tetramethylammonium), heteroarylium, (e.g., N-methyl-pyridinium), basic nitrogens which are protonated at physiological pH, and the like. Cationic groups thus encompass positively charged nitrogen-containing groups, as well as basic nitrogens which are protonated at physiologic pH.

Whenever referred to, the term “heteroatom” means O, S or N, selected on an independent basis.

Whenever referred to, halogen and “halo” refer to bromine, chlorine, fluorine and iodine. Whenever referred to, alkoxy refers to C1-C4 alkyl-O—, with the alkyl group optionally substituted as described herein.

Whenever referred to, guanidinyl refers to the group: H2NC(NH)NH—.

Whenever referred to, carbamimidoyl refers to the group: H2NC(NH)—.

Whenever referred to, ureido refers to the group: H2NC(O)NH—.

When a group is termed “substituted”, unless otherwise indicated, this means that the group contains from 1 to 4 substituents thereon. With respect to R, Ra, Rb and Rc, the substituents available on alkyl groups are selected from the values of Rd. Many of the variable groups are optionally substituted with up to four Ri groups. With respect to Re, Rf and Rg, when these variables represent substituted alkyl, the substituents available thereon are selected from the values of Ri.

When a functional group is termed “protected”, this means that the group is in modified form to preclude undesired side reactions at the protected site. Suitable protecting groups for the compounds of the present invention will be recognized from the present application taking into account the level of skill in the art, and with reference to standard textbooks, such as Greene, T. W. et al. Protective Groups in Organic Synthesis Wiley, New York (1991). Examples of suitable protecting groups are contained throughout the specification.

Whenever present, in some of the embodiments of the present invention, M can be used to denote a readily removable carboxyl protecting group, and/or P can be used to denote a hydroxyl which is protected by a hydroxyl-protecting group. Such conventional protecting groups consist of known groups which are used to protectively block the hydroxyl or carboxyl group during the synthesis procedures described herein. These conventional blocking groups are readily removable, i.e., they can be removed, if desired, by procedures which will not cause cleavage or other disruption of the remaining portions of the molecule. Such procedures include chemical and enzymatic hydrolysis, treatment with chemical reducing or oxidizing agents under mild conditions, treatment with a transition metal catalyst and a nucleophile and catalytic hydrogenation.

Examples of carboxyl protecting groups include allyl, benzhydryl, 2-naphthylmethyl, benzyl, silyl such as t-butyldimethylsilyl (TBDMS), phenacyl, p-methoxybenzyl, o-nitrobenzyl, p-methoxyphenyl, p-nitrobenzyl, 4-pyridylmethyl and t-butyl.

Examples of suitable C-6 hydroxyethyl protecting groups include triethylsilyl, t-butyldimethylsilyl, o-nitrobenzyl-oxycarbonyl, p-nitrobenzyloxycarbonyl, benzyloxycarbonyl, allyloxycarbonyl, t-butyloxycarbonyl, 2,2,2-trichloroethyloxy-carbonyl and the like.

Whenever present, with respect to —CO2M, which is attached to the carbapenem nucleus at position 3, this represents a carboxylic acid group (M represents H), a carboxylate anion (M represents a negative charge), a pharmaceutically acceptable ester (M represents an ester forming group) or a carboxylic acid protected by a protecting group (M represents a carboxyl protecting group).

Whenever present, the pharmaceutically acceptable salts referred to above may take the form —COOM, where M is a negative charge, which is balanced by a counterion, e.g., an alkali metal cation such as sodium or potassium. Other pharmaceutically acceptable counterions may be calcium, magnesium, zinc, ammonium, or alkylammonium cations such as tetramethylammonium, tetrabutylammonium, choline, triethylhydroammonium, meglumine, triethanolhydroammonium, etc.

Whenever present, the pharmaceutically acceptable salts referred to above also include acid addition salts. Thus, the Formula I compounds can be used in the form of salts derived from inorganic or organic acids. Included among such salts are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate and undecanoate.

Whenever referred to, the pharmaceutically acceptable esters are such as would be readily apparent to a medicinal chemist, and include, for example, those described in detail in U.S. Pat. No. 4,309,438. Included within such pharmaceutically acceptable esters are those which are hydrolyzed under physiological conditions, such as pivaloyloxymethyl, acetoxymethyl, phthalidyl, indanyl and methoxymethyl, and others described in detail in U.S. Pat. No. 4,479,947. These are also referred to as “biolabile esters”.

Whenever referred to, biolabile esters are biologically hydrolizable, and may be suitable for oral administration, due to good absorption through the stomach or intestinal mucosa, resistance to gastric acid degradation and other factors. Examples of biolabile esters include compounds in which M represents an alkoxyalkyl, alkylcarbonyloxyalkyl, alkoxycarbonyloxyalkyl, cycloalkoxyalkyl, alkenyloxyalkyl, aryloxyalkyl, alkoxyaryl, alkylthioalkyl, cycloalkylthioalkyl, alkenylthioalkyl, arylthioalkyl or alkylthioaryl group. These groups can be substituted in the alkyl or aryl portions thereof with acyl or halo groups. The following M species are examples of biolabile ester forming moieties: acetoxymethyl, 1-acetoxyethyl, 1-acetoxypropyl, pivaloyloxymethyl, 1-isopropyloxycarbonyloxyethyl, 1-cyclohexyloxycarbonyloxyethyl, phthalidyl and (2-oxo-5-methyl-1,3-dioxolen-4-yl)methyl.

Whenever present, L- can be present or absent as necessary to maintain the appropriate charge balance. When present, L- represents a pharmaceutically acceptable counterion. Most anions derived from inorganic or organic acids are suitable. Representative examples of such counterions are the following: acetate, adipate, aminosalicylate, anhydromethylenecitrate, ascorbate, aspartate, benzoate, benzenesulfonate, bromide, citrate, camphorate, camphorsulfonate, chloride, estolate, ethanesulfonate, fumarate, glucoheptanoate, gluconate, glutamate, lactobionate, malate, maleate, mandelate, methanesulfonate, pantothenate, pectinate, phosphate/diphosphate, polygalacturonate, propionate, salicylate, stearate, succinate, sulfate, tartrate and tosylate. Other suitable anionic species will be apparent to the ordinarily skilled chemist.

Likewise, when L- represents a specie with more than one negative charge, such as malonate, tartrate or ethylenediamine-tetraacetate (EDTA), an appropriate number of carbapenem molecules can be found in association therewith to maintain the overall charge balance and neutrality.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of refers to those elements required for a given embodiment. The term permits the presence of elements that do not materially affect the basic and novel or functional characteristic(s) of that embodiment of the invention.

As used herein, the term “desirable property” refers to any attributes of a biologies that is significant with respect to the biologies' action as a therapeutics or biologically active agent. Such desirable properties include, for example, blood circulation life, shelf-life, hydrophobicity or hydrophilicity, biological activity, bioavailability, cytotoxicity, non-immunogenicity, or conformational properties, etc.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

### Random Conjugate Molecule

In one aspect of the present invention, it is provided a random conjugate molecule (CMN), comprising a formula of CG-X-N,

wherein:
CG is at least one chemical group,
X is a linking group, and
N is NELL-1 protein or peptide.

In some embodiments of the invention molecule, optionally in combination with any or all of the various embodiments disclosed herein, the at least one chemical group is an alkyl group, an aryl group, an acyl group, a leaving group, a polymer, or a peptide, or a combination thereof.

In some embodiments of the invention molecule, optionally in combination with any or all of the various embodiments disclosed herein, the chemical group is poly(ethylene glycol) (PEG), poly(ethylene oxide) (PEO), poly(propylene glycol) (PPG), or poly(propylene oxide) (PPO).

In some embodiments of the invention molecule, optionally in combination with any or all of the various embodiments disclosed herein, the chemical group is selected from heparin sulfate, glycopolymers, zwitterionic polymers, hyperbranched polymers, polymers containing unnatural amino acids, linkers to lysine or cysteine, or peptide sequences that modify NN or CMN interactions with ECM, target cells, immune cells, and hepatocytes.

In some embodiments of the invention molecule, optionally in combination with any or all of the various embodiments disclosed herein, the linking group comprises a responsive linker that degrades on demand to external stimuli.

In some embodiments of the invention molecule, optionally in combination with any or all of the various embodiments disclosed herein, the linking group provides a linking reaction that is azide-alkyne, azide-BMCO, or Tetrazine-TCO type reactions.

In some embodiments of the invention molecule, optionally in combination with any or all of the various embodiments disclosed herein, the linking group comprises a linker to lysine or cysteine.

In some embodiments of the invention molecule, optionally in combination with any or all of the various embodiments disclosed herein, the linking group comprises a natural enzyme.

In some embodiments of the invention molecule, optionally in combination with any or all of the various embodiments disclosed herein, the at least one chemical group imparts at least one desirable property to the NELL-1 protein such that the CMN is significantly improved in the at least one desirable property relative to a naked NELL-1 protein without chemical modification (NN).

In some embodiments of the invention molecule, optionally in combination with any or all of the various embodiments of the present invention, the at least one desirable property is selected from the group consisting of blood circulation life, shelf-life, hydrophobicity or hydrophilicity, biological activity, bioavailability, cytotoxicity, non-immunogenicity, or conformational properties, etc.

As used herein, the term “significantly” in connection with the phrase “significantly improved” shall mean “statistically significant” and, in certain embodiments, can mean an improvement of 10% or above, 20% or above, 30% or above, 40% or above, 50% or above, 60% or above, 70% or above, 80% or above, 90% or above, 100% or above, 200% or above, 300% or above, 400% or above, 500% or above, 600% or above, 700% or above, 800% or above, 900% or above, or 1000% or above.

### NELL-1 Protein

NEL-like molecule-1 (NELL-1) protein is widely studied in bone regeneration as an osteogenic growth factor with higher specificity to osteoblast cells compared to the growth factors currently used such as BMP-2 [1-4]. NELL-1 is a secreted homotrimer protein with molecular weight up to 400 KDa. The subunit of NELL-1 contains 810 amino acids and a molecular weight of about 90 KDa before N-glycosylation and oligomerisation [5]. Previous studies suggested that NELL-1 can specifically modulate the osteochondral lineage and induce bone formation in various kinds of animal models from rodents to sheep [1,6]. Recently, Kwak et. al have demonstrated that the locally intramedullary application of NELL-1 in the femurs of ovariectomy (OVX)-induced osteoporotic female rats could enhance rat bone quality and prevent osteoporosis [7]. In vivo studies further indicated that the deficit of Nell-1 gene or loss NELL-1 function may contribute to the development of osteoporosis in animal and clinical researches [8, 9]. These studies suggest that the NELL-1 protein has potential to be used for treatment of osteoporosis by simple intravenous injection.

NELL-1 is often applied in local tissues (spine, femur, calvaria, etc) by being loaded onto various carriers including tricalcium phosphate (TCP) particles [10], demineralized bone matrix (DBM), and PLGA scaffold [2, 10]. But for the treatment of osteoporosis disease, it is necessary to be administered by intravenous injection that can lead to systemic functional improvement of bone quality. However, due to the rapid clearance of native protein drug in vivo, high dose and frequent administration usually have to be adopted to achieve therapeutic benefit. This can lead to high treatment cost and low patient compliance in chronic treatment. The short circulation time of NELL-1 in vivo could be one of the main limitations for the practical application of systemic therapy. Therefore, the main purpose of the present study was to extend the circulation time of NELL-1 in vivo by chemically modifying its molecular structure. Currently, one of the most popular technologies to prolong the half-life time of protein is to use water soluble polymers as a macromolecular carrier. As it is approved for human use by FDA, the non-toxic PEG molecule is widely used in numerous biomedical applications [11-13]. It is a water soluble polymer with excellent biocompatibility but without immunogenicity. PEG is commercially available in a wide range of molecular weights, which is particularly appropriate for the chemical attachment to proteins with various molecular weights. So it was chosen to conjugate with NELL-1 protein in the current study.

The methods of chemical modification of protein with PEG can be achieved by random conjugation. To the best of our knowledge, no reports have been made on the PEGylation of NELL-1, a huge protein with the Mw much larger than all other proteins that have been PEGylated to date. In one study of an embodiment of the present invention, we PEGylated NELL-1 by random conjugation using three different PEG sizes (5, 20, 40 kDa). The PEGylated NELL-1 was synthesized using chemically activated PEG-N-hydroxysuccinimide (PEG-NHS) for conjugation with the amine group in lysine residue located at the surface of NELL-1. NHS was chosen for amine coupling reactions due to its high reactivity in bio-conjugation synthesis at physiological pH [17]. For each PEGylated NELL-1, the PEG modification degree, thermal stability, and cytotoxicity were determined. The in vitro bioactivity study of NELL-PEG was also evaluated in two primary cell lines, human perivascular stem cells (hPSC) and mouse calvarial osteoblast cells. Subsequently, the pharmacokinetic behavior of the PEGylated NELL-1 was examined in mice.

### Bioconjugation to NELL-1 Protein

Some examples of the modifying chemical group is an alkyl group, an aryl group, an acyl group, a leaving group, a polymer, or a peptide, or a combination thereof.

Some further examples of the chemical group is poly(ethylene glycol) (PEG), poly(ethylene oxide) (PEO), poly(propylene glycol) (PPG), or poly(propylene oxide) (PPO).

Still some further examples of the chemical group is heparin sulfate (different from heparin), glycopolymers, zwitterionic polymers, hyperbranched polymers, polymers containing un-natural amino acids.

Bioconjugation to NELL-1 can be achieved via established reactions. For example, conjugation can occur via azide-alkyne, azide-BMCO, and Tetrazine-TCO type reactions. Other types of possible linkers to lysine, cysteine, etc. are described in Craig S. McKayl and M. G. Finn, 2014, supra, the teaching of which is incorporate herein by reference.

Some further examples of chemical group conjugation to NELL-1 include, for example, conjugating peptide sequences to Nell or to the CMN to modulate interactions with ECM, target cells, immune cells, and hepatocytes, etc.

As further examples of conjugation to NELL-1 protein include, for example, inserting responsive linkers that degrade on demand to external stimuli (pH, heat, specific wavelength, ultrasound, electric current, magnetic simulation, biomolecules and proteins). In such examples, one can allow a CMN to circulate in blood in a protected form systemically, but at a selected site (e.g., the hip), he/she can stimulate the linker to degrade locally by delivering local stimuli for a desired period of time (e.g., 5 minutes a day, etc.) Alternatively, for spinal fusion, NELL-1 protein is protected as a CMN and administered to the spinal fusion site and allowed to slowly diffuse until a stimuli is delivered to convert CMN to NELL-1.

Still, as further examples, a natural enzyme is used to link a protective or biofunctionalized coating onto Nell. For example, one can use Factor XIII which crosslinks fibrinogen at specific sites. By encoding the Factor XIII peptide sequences into Nell, or conjugating the peptides onto Nell surface and the protective coating material, one can then use Factor XIII to conjugate the protective coating onto Nell. Besides Factor XIII, many natural enzymes that act on natural proteins, and natural metabolic precursors can work. For example sortase A can be used by encoding or conjugating a short peptide sequence onto Nell.

### Method of Preparation

In a further aspect of the present invention, it is provided a method of preparing a random conjugate molecule (CMN) of a formula of CG-X-N, comprising:

a) providing a NELL-1 protein or peptide (N),

b) providing a chemical compound comprising a chemical group (CG),

c) providing a linking group that comprises a linker (X), and

d) bringing the CG into contact with the NELL-1 protein or peptide to cause conjugation between the chemical group and the NELL-1 protein or peptide to occur to form the CMN.

In some embodiments of the invention method, optionally in combination with any or all of the various embodiments disclosed herein, the at least one chemical group is an alkyl group, an aryl group, an acyl group, a leaving group, a polymer, or a peptide, or a combination thereof.

In some embodiments of the invention method, optionally in combination with any or all of the various embodiments disclosed herein, the chemical group is poly(ethylene glycol) (PEG), poly(ethylene oxide) (PEO), poly(propylene glycol) (PPG), or poly(propylene oxide) (PPO).

In some embodiments of the invention method, optionally in combination with any or all of the various embodiments disclosed herein, the chemical group is selected from heparin sulfate, glycopolymers, zwitterionic polymers, hyperbranched polymers, polymers containing unnatural amino acids, linkers to lysine or cysteine, or peptide sequences that modify NN or CMN interactions with ECM, target cells, immune cells, and hepatocytes.

In some embodiments of the invention method, optionally in combination with any or all of the various embodiments disclosed herein, the linking group comprises a responsive linker that degrades on demand to external stimuli.

In some embodiments of the invention method, optionally in combination with any or all of the various embodiments disclosed herein, the linking group provides a linking reaction that is azide-alkyne, azide-BMCO, or Tetrazine-TCO type reactions.

In some embodiments of the invention method, optionally in combination with any or all of the various embodiments disclosed herein, the linking group comprises a linker to lysine or cysteine.

In some embodiments of the invention method, optionally in combination with any or all of the various embodiments disclosed herein, the linking group comprises a natural enzyme.

In some embodiments of the invention method, optionally in combination with any or all of the various embodiments disclosed herein, the at least one chemical group imparts at least one desirable property to the NELL-1 protein such that the CMN is significantly improved in the at least one desirable property relative to a naked NELL-1 protein without chemical modification (NN).

In some embodiments of the invention method, optionally in combination with any or all of the various embodiments of the present invention, the at least one desirable property is selected from the group consisting of blood circulation life, shelf-life, hydrophobicity or hydrophilicity, biological activity, bioavailability, cytotoxicity, non-immunogenicity, or conformational properties, etc.

### Compositions

In another aspect of the present invention, it is provided a composition comprising a random conjugate molecule (CMN) of a formula of CG-X-N,

wherein:
CG is at least one chemical group,
X is a linking group, and
N is NELL-1 protein or peptide.

In some embodiments of the invention composition, optionally in combination with any or all of the various embodiments disclosed herein, the at least one chemical group is an alkyl group, an aryl group, an acyl group, a leaving group, a polymer, or a peptide, or a combination thereof.

In some embodiments of the invention composition, optionally in combination with any or all of the various embodiments disclosed herein, the chemical group is poly(ethylene glycol) (PEG), poly(ethylene oxide) (PEO), poly(propylene glycol) (PPG), or poly(propylene oxide) (PPO).

In some embodiments of the invention composition, optionally in combination with any or all of the various embodiments disclosed herein, the chemical group is selected from heparin sulfate, glycopolymers, zwitterionic polymers, hyperbranched polymers, polymers containing unnatural amino acids, linkers to lysine or cysteine, or peptide sequences that modify NN or CMN interactions with ECM, target cells, immune cells, and hepatocytes.

In some embodiments of the invention composition, optionally in combination with any or all of the various embodiments disclosed herein, the linking group comprises a responsive linker that degrades on demand to external stimuli.

In some embodiments of the invention composition, optionally in combination with any or all of the various embodiments disclosed herein, the linking group provides a linking reaction that is azide-alkyne, azide-BMCO, or Tetrazine-TCO type reactions.

In some embodiments of the invention composition, optionally in combination with any or all of the various embodiments disclosed herein, the linking group comprises a linker to lysine or cysteine.

In some embodiments of the invention composition, optionally in combination with any or all of the various embodiments disclosed herein, the linking group comprises a natural enzyme.

In some embodiments of the invention composition, optionally in combination with any or all of the various embodiments disclosed herein, the at least one chemical group imparts at least one desirable property to the NELL-1 protein such that the CMN is significantly improved in the at least one desirable property relative to a naked NELL-1 protein without chemical modification (NN).

In some embodiments of the invention composition, optionally in combination with any or all of the various embodiments disclosed herein, the composition further comprises a pharmaceutically acceptable carrier.

In some embodiments of the invention composition, optionally in combination with any or all of the various embodiments disclosed herein, the composition is a formulation for systemic or local delivery.

In some embodiments of the invention composition, optionally in combination with any or all of the various embodiments of the present invention, the at least one desirable property is selected from the group consisting of blood circulation life, shelf-life, hydrophobicity or hydrophilicity, biological activity, bioavailability, cytotoxicity, non-immunogenicity, or conformational properties, etc.

### Methods of Fabrication

In a further aspect of the present invention, it is provided a method of forming a composition, comprising:

providing an amount of a random conjugate molecule (CMN) of a formula of CG-X-N, and

forming the composition,

wherein:
CG is at least one chemical group,
X is a linking group, and
N is NELL-1 protein or peptide.

In some embodiments of the invention method, optionally in combination with any or all of the various embodiments disclosed herein, the at least one chemical group is an alkyl group, an aryl group, an acyl group, a leaving group, a polymer, or a peptide, or a combination thereof.

In some embodiments of the invention method, optionally in combination with any or all of the various embodiments disclosed herein, the chemical group is poly(ethylene glycol) (PEG), poly(ethylene oxide) (PEO), poly(propylene glycol) (PPG), or poly(propylene oxide) (PPO).

In some embodiments of the invention method, optionally in combination with any or all of the various embodiments disclosed herein, the chemical group is selected from heparin sulfate, glycopolymers, zwitterionic polymers, hyperbranched polymers, polymers containing unnatural amino acids, linkers to lysine or cysteine, or peptide sequences that modify NN or CMN interactions with ECM, target cells, immune cells, and hepatocytes.

In some embodiments of the invention method, optionally in combination with any or all of the various embodiments disclosed herein, the linking group comprises a responsive linker that degrades on demand to external stimuli.

In some embodiments of the invention method, optionally in combination with any or all of the various embodiments disclosed herein, the linking group provides a linking reaction that is azide-alkyne, azide-BMCO, or Tetrazine-TCO type reactions.

In some embodiments of the invention method, optionally in combination with any or all of the various embodiments disclosed herein, the linking group comprises a linker to lysine or cysteine.

In some embodiments of the invention method, optionally in combination with any or all of the various embodiments disclosed herein, the linking group comprises a natural enzyme.

In some embodiments of the invention method, optionally in combination with any or all of the various embodiments disclosed herein, the at least one chemical group imparts at least one desirable property to the NELL-1 protein such that the CMN is significantly improved in the at least one desirable property relative to a naked NELL-1 protein without chemical modification (NN).

In some embodiments of the invention method, optionally in combination with any or all of the various embodiments disclosed herein, the composition further comprises a pharmaceutically acceptable carrier.

In some embodiments of the invention method, optionally in combination with any or all of the various embodiments disclosed herein, the composition is a formulation for systemic or local delivery.

In some embodiments of the invention method, optionally in combination with any or all of the various embodiments of the present invention, the at least one desirable property is selected from the group consisting of blood circulation life, shelf-life, hydrophobicity or hydrophilicity, biological activity, bioavailability, cytotoxicity, non-immunogenicity, or conformational properties, etc.

### Carriers

The present invention involves compositions useful for practicing the therapeutic methods described herein. Therapeutic compositions contain a physiologically tolerable carrier together with an active agent as described herein, dissolved or dispersed therein as an active ingredient. In a preferred embodiment, the therapeutic composition is not immunogenic when administered to a mammal or human patient for therapeutic purposes. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

Pharmaceutically acceptable carrier is well known in the art. Examples of such carrier includes, e.g., salient, for liquid or suspension formulations, natural or synthetic polymeric materials for burst or sustained release formulations or targeted delivery formulations. Some examples of the carriers are further described in detail below.

### Polymeric Materials

In some embodiments, the carrier disclosed herein can be a polymeric material Exemplary polymeric material that can be used here include but are not limited to a biocompatible or bioabsorbable polymer that is one or more of poly(DL-lactide), poly(L-lactide), poly(L-lactide), poly(L-lactide-co-D,L-lactide), polymandelide, polyglycolide, poly(lactide-co-glycolide), poly(D,L-lactide-co-glycolide), poly(L-lactide-co-glycolide), poly(ester amide), poly(ortho esters), poly(glycolic acid-co-trimethylene carbonate), poly(D,L-lactide-co-trimethylene carbonate), poly(trimethylene carbonate), poly(lactide-co-caprolactone), poly(glycolide-co-caprolactone), poly(tyrosine ester), polyanhydride, derivatives thereof. In some embodiments, the polymeric material comprises a combination of these polymers.

In some embodiments, the polymeric material comprises poly(D,L-lactide-co-glycolide). In some embodiments, the polymeric material comprises poly(D,L-lactide). In some embodiments, the polymeric material comprises poly(L-lactide). [0065] Additional exemplary polymers include but are not limited to poly(D-lactide) (PDLA), polymandelide (PM), polyglycolide (PGA), poly(L-lactide-co-D,L-lactide) (PLDLA), poly(D,L-lactide) (PDLLA), poly(D,L-lactide-co-glycolide) (PLGA) and poly(L-lactide-co-glycolide) (PLLGA). With respect to PLLGA, the stent scaffolding can be made from PLLGA with a 25 mole % of GA between 5-15 mol %. The PLLGA can have a mole % of (LA:GA) of 85:15 (or a range of 82:18 to 88:12), 95:5 (or a range of 93:7 to 97:3), or commercially available PLLGA products identified as being 85:15 or 95:5 PLLGA. The examples provided above are not the only polymers that may be used. Many other examples can be provided, such as those found in Polymeric Biomaterials, second edition, edited by Severian Dumitriu; chapter 4.

In some embodiments, polymers that are more flexible or that have a lower modulus that those mentioned above may also be used. Exemplary lower modulus bioabsorbable polymers include, polycaprolactone (PCL), poly(trimethylene carbonate) (PTMC), polydioxanone (PDO), poly(3-hydrobutyrate) (PHB), poly(4-hydroxybutyrate) (P4HB), poly(hydroxyalkanoate) (PHA), and poly(butylene succinate), and blends and copolymers thereof.

In exemplary embodiments, higher modulus polymers such as PLLA or PLLGA may be blended with lower modulus polymers or copolymers with PLLA or PLGA. The blended lower modulus polymers result in a blend that has a higher fracture toughness than the high modulus polymer. Exemplary low modulus copolymers include poly(L-lactide)-b-polycaprolactone (PLLA-b-PCL) or poly(L-lactide)-co-polycaprolactone (PLLA-co-PCL). The composition of a blend can include 1-5 wt % of low modulus polymer.

More exemplary polymers include but are not limited to at least partially alkylated polyethyleneimine (PEI); at least partially alkylated poly(lysine); at least partially alkylated polyornithine; at least partially alkylated poly(amido amine), at least partially alkylated homo- and co-polymers of vinylamine; at least partially alkylated acrylate containing aminogroups, copolymers of vinylamine containing aminogroups with hydrophobic monomers, copolymers of acrylate containing aminogroups with hydrophobic monomers, and amino containing natural and modified polysaccharides, polyacrylates, polymethacryates, polyureas, polyurethanes, polyolefins, polyvinylhalides, polyvinylidenehalides, polyvinylethers, polyvinylaromatics, polyvinylesters, polyacrylonitriles, alkyd resins, polysiloxanes and epoxy resins, and mixtures thereof. [0069] Additional examples of biocompatible biodegradable polymers include, without limitation, polycaprolactone, poly(L-lactide), poly(D,L-lactide), poly(D,L-lactide-co-PEG) block copolymers, poly(D,L-lactide-co-trimethylene carbonate), poly(lactide-co-glycolide), polydioxanone (PDS), polyorthoester, polyanhydride, poly(glycolic acid-co-trimethylene carbonate), polyphosphoester, polyphosphoester urethane, poly(amino acids), polycyanoacrylates, poly(trimethylene carbonate), poly(iminocarbonate), polycarbonates, polyurethanes, polyalkylene oxalates, polyphosphazenes, PHA-PEG, and combinations thereof. The PHA may include poly(a-hydroxyacids), poly(P-hydroxyacid) such as poly(3-hydroxybutyrate) (PHB), poly(3-hydroxybutyrate-co-valerate) (PHBV), poly(3-hydroxyproprionate) (PHP), poly(3-hydroxyhexanoate) (PHH), or poly(4-hydroxyacid) such as poly poly(4-hydroxybutyrate), poly(4-hydroxyvalerate), poly(4-hydroxyhexanoate), poly(hydroxyvalerate), poly(tyrosine carbonates), poly(tyrosine arylates), poly(ester amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, poly(D,L-lactide), poly(L-lactide), polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(trimethylene carbonate), poly(iminocarbonate), polyphosphazenes, silicones, polyesters, polyolefms, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters) (e.g. poly(ethylene oxide-co-lactic acid) (PEO/PLA)), polyalkylene oxides such as poly(ethylene oxide), poly(propylene oxide), poly(ether ester), polyalkylene oxalates, phosphoryl choline containing polymer, choline, poly(aspirin), polymers and co-polymers of hydroxyl bearing monomers such as 2-hydroxyethyl methacrylate (HEMA), hydroxypropyl methacrylate (HPMA), hydroxypropylmethacrylamide, PEG acrylate (PEGA), PEG methacrylate, methacrylate polymers containing 2-methacryloyloxyethyl-phosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate), MED610, poly(methyl methacrylate)-PEG (PMMA-PEG), polydimethylsiloxane-co-PEG (PDMS-PEG), poly(vinylidene fiuoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as collagen, chitosan, alginate, fibrin, fibrinogen, cellulose, starch, dextran, dextrin, hyaluronic acid, fragments and derivatives of hyaluronic acid, heparin, fragments and derivatives of heparin, glycosamino glycan (GAG), GAG derivatives, polysaccharide, elastin, elastin protein mimetics, or combinations thereof.

In some embodiments, polyethylene is used to construct at least a portion of the device. For example, polyethylene can be used in an orthopedic implant on a surface that is designed to contact another implant, as such in a joint or hip replacement. Polyethylene is very durable when it comes into contact with other materials. When a metal implant moves on a polyethylene surface, as it does in most joint replacements, the contact is very smooth and the amount of wear is minimal. Patients who are younger or more active may benefit from polyethylene with even more resistance to wear. This can be accomplished through a process called crosslinking, which creates stronger bonds between the elements that make up the polyethylene. The appropriate amount of crosslinking depends on the type of implant. For example, the surface of a hip implant may require a different degree of crosslinking than the surface of a knee implant.

Additional examples of polymeric materials can be found, for example, in U.S. Pat. No. 6,127,448 to Domb, US Pat. Pub. No. 2004/0148016 by Klein and Brazil, US Pat. Pub. No. 2009/0169714 by Burghard et al, U.S. Pat. No. 6,406,792 to Briquet et al, US Pat. Pub. No. 2008/0003256 by Martens et al, each of which is hereby incorporated by reference herein in its entirety.

### Methods of Use

In still a further aspect of the present invention, it is provided method of treating or ameliorating a condition in a subject, comprising administering to the subject a random conjugate molecule (CMN) of a formula of CG-X-N,

wherein:
CG is at least one chemical group,
X is a linking group, and
N is NELL-1 protein or peptide.

In some embodiments of the invention method, optionally in combination with any or all of the various embodiments disclosed herein, the at least one chemical group is an alkyl group, an aryl group, an acyl group, a leaving group, a polymer, or a peptide, or a combination thereof.

In some embodiments of the invention molecule, optionally in combination with any or all of the various embodiments disclosed herein, the chemical group is poly(ethylene glycol) (PEG), poly(ethylene oxide) (PEO), poly(propylene glycol) (PPG), or poly(propylene oxide) (PPO).

In some embodiments of the invention method, optionally in combination with any or all of the various embodiments disclosed herein, the chemical group is selected from heparin sulfate, glycopolymers, zwitterionic polymers, hyperbranched polymers, polymers containing unnatural amino acids, linkers to lysine or cysteine, or peptide sequences that modify NN or CMN interactions with ECM, target cells, immune cells, and hepatocytes.

In some embodiments of the invention method, optionally in combination with any or all of the various embodiments disclosed herein, the linking group comprises a responsive linker that degrades on demand to external stimuli.

In some embodiments of the invention method, optionally in combination with any or all of the various embodiments disclosed herein, the linking group provides a linking reaction that is azide-alkyne, azide-BMCO, or Tetrazine-TCO type reactions.

In some embodiments of the invention method, optionally in combination with any or all of the various embodiments disclosed herein, the linking group comprises a linker to lysine or cysteine.

In some embodiments of the invention method, optionally in combination with any or all of the various embodiments disclosed herein, the linking group comprises a natural enzyme.

In some embodiments of the invention method, optionally in combination with any or all of the various embodiments disclosed herein, the at least one chemical group imparts at least one desirable property to the NELL-1 protein such that the CMN is significantly improved in the at least one desirable property relative to a naked NELL-1 protein without chemical modification (NN).

In some embodiments of the invention method, optionally in combination with any or all of the various embodiments of the present invention, the at least one desirable property is selected from the group consisting of blood circulation life, shelf-life, hydrophobicity or hydrophilicity, biological activity, bioavailability, cytotoxicity, non-immunogenicity, or conformational properties, etc.

In some embodiments of the invention method, optionally in combination with any or all of the various embodiments of the present invention, the CMN is included in a composition that comprises the CMN and a pharmaceutically acceptable carrier.

In some embodiments of the invention method, optionally in combination with any or all of the various embodiments of the present invention, the composition is a formulation for local or systemic delivery.

In some embodiments of the invention method, optionally in combination with any or all of the various embodiments of the present invention, the bone condition is osteoporosis.

In some embodiments of the invention method, optionally in combination with any or all of the various embodiments of the present invention, the bone condition is osteoporosis.

In some embodiments of the invention method, optionally in combination with any or all of the various embodiments of the present invention, the bone condition is bone fracture or intervertebral disc disease or injury.

In some embodiments of the invention method, optionally in combination with any or all of the various embodiments of the present invention, wherein administering comprises local or systemic administration.

In some embodiments of the invention method, optionally in combination with any or all of the various embodiments of the present invention, the subject is a human being.

### Dosage and Administration

The dosage can be determined by one of skill in the art and can also be adjusted by the individual physician in the event of any complication. Typically, the dosage ranges from 0.0005 mg/kg body weight to 1 g/kg body weight. In some embodiments, the dosage range is from 0.001 mg/kg body weight to 0.5 g/kg body weight, from 0.0005 mg/kg body weight to 0.1 g/kg body weight, from 0.001 mg/kg body weight to 0.05 g/kg body weight.

As another alternative, dosage are selected for localized delivery and are not necessary selected to body weight or to achieve a certain serum level, but to achieve a localized effect, e.g., as for a localized injection, implantation or other localized administration to the eye.

Administration of the doses recited above can be repeated for a limited period of time. In some embodiments, the doses are given once a day, or multiple times a day, for example but not limited to three times a day. In a preferred embodiment, the doses recited above are administered daily for several weeks or months. The duration of treatment depends upon the subject's clinical progress and responsiveness to therapy. Continuous, relatively low maintenance doses are contemplated after an initial higher therapeutic dose.

Agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art. It is preferred that the agents for the methods described herein are administered topically to the eye. For the treatment of tumors, the agent can be administered systemically, or alternatively, can be administered directly to the tumor e.g., by intratumor injection or by injection into the tumor's primary blood supply.

Therapeutic compositions containing at least one agent disclosed herein can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology. Antibody-based or non-antibody-based targeting moieties can be employed to deliver a ligand or the inhibitor to a target site. Preferably, a natural binding agent for an unregulated or disease associated antigen is used for this purpose.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

An agent may be adapted for catheter-based delivery systems including coated balloons, slow-release drug-eluting stents or other drug-eluting formats, microencapsulated PEG liposomes, or nanobeads for delivery using direct mechanical intervention with or without adjunctive techniques such as ultrasound.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

The following examples illustrate rather than limit the embodiments of the present invention.

### Example 1: Preserved Bioactivity of PEGylated NELL-1 and its Long Circulation in Mice for Potential Osteoporosis Therapy

### 1. Introduction

NEL-like molecule-1 (NELL-1) protein is widely studied in bone regeneration as an osteogenic growth factor with higher specificity to osteoblast cells compared to the growth factors currently used such as BMP-2 [1-4]. NELL-1 is a secreted homotrimer protein with molecular weight up to 400 KDa. The subunit of NELL-1 contains 810 amino acids and a molecular weight of about 90 KDa before N-glycosylation and oligomerisation [5]. Previous studies suggested that NELL-1 can specifically modulate the osteochondral lineage and induce bone formation in various kinds of animal models from rodents to sheep [1,6]. Recently, Kwak et. al have demonstrated that the locally intramedullary application of NELL-1 in the femurs of ovariectomy (OYX)-induced osteoporotic female rats could enhance rat bone quality and prevent osteoporosis [7]. In vivo studies further indicated that the deficit of Nell-1 gene or loss NELL-1 function may contribute to the development of osteoporosis in animal and clinical researches [8, 9]. These studies suggest that the NELL-1 protein has potential to be used for treatment of osteoporosis by simple intravenous injection.

NELL-1 is often applied in local tissues (spine, femur, calvaria, etc) by being loaded onto various carriers including tricalcium phosphate (TCP) particles [10], demineralized bone matrix (DBM), and PLGA scaffold [2, 10]. But for the treatment of osteoporosis disease, it is necessary to be administered by intravenous injection that can lead to systemic functional improvement of bone quality. However, due to the rapid clearance of native protein drug in vivo, high dose and frequent administration usually have to be adopted to achieve therapeutic benefit. This can lead to high treatment cost and low patient compliance in chronic treatment. The short circulation time of NELL-1 in vivo could be one of the main limitations for the practical application of systemic therapy. Therefore, the main purpose of the present study was to extend the circulation time of NELL-1 in vivo by chemically modifying its molecular structure. Currently, one of the most popular technologies to prolong the half-life time of protein is to use water soluble polymers as a macromolecular carrier. As it is approved for human use by FDA, the non-toxic PEG molecule is widely used in numerous biomedical applications [11-13]. It is a water soluble polymer with excellent biocompatibility but without immunogenicity. PEG is commercially available in a wide range of molecular weights, which is particularly appropriate for the chemical attachment to proteins with various molecular weights. So it was chosen to conjugate with NELL-1 protein in the current study.

The methods of chemical modification of protein with PEG can be divided into two categories: site-specific conjugation and random conjugation. The site-specific conjugation method can produce better defined products using an N-terminal amine-specific or cysteine-specific PEGylation reaction. The N-terminal PEGylation often uses a PEGylating reagent with relatively low reactivity (such as PEG-aldehyde), since a high reactive PEG reagent will lead to an evident degree of lysine coupling [14]. Therefore, incomplete PEGylation and low yield were associated with this method. Cysteine-specific PEGylation can get a higher yield, but the problem is that the cysteine group of reduced form is rarely available in proteins because it is usually involved in disulfide bridges. Even naturally present, the cysteine group often plays an important role in protein structure or activity, and the modification on it could lead to significantly reduced or lost bioactivity [15]. The approach of random conjugation is often used as the first method in many new PEG-protein studies since it is conventional and convenient. This could result in complex mixtures of various PEG-conjugate isomers differing both in the number of PEG molecules and the site of linking [16], but the advantage is that it is simple and can achieve sound PEG-conjugates with high yields. Furthermore, the PEG conjugate can be purified to produce a homogenous product.

To the best of our knowledge, no reports have been made on the PEGylation of NELL-1, a huge protein with the Mw much larger than all other proteins that have been PEGylated to date. In the present study, we PEGylated NELL-1 by random conjugation using three different PEG sizes (5, 20, 40 kDa). The PEGylated NELL-1 was synthesized using chemically activated PEG-N-hydroxysuccinimide (PEG-NHS) for conjugation with the amine group in lysine residue located at the surface of NELL-1. NHS was chosen for amine coupling reactions due to its high reactivity in bio-conjugation synthesis at physiological pH [17]. For each PEGylated NELL-1, the PEG modification degree, thermal stability, and cytotoxicity were determined. The in vitro bioactivity study of NELL-PEG was also evaluated in two primary cell lines, human perivascular stem cells (hPSC) and mouse calvarial osteoblast cells. Subsequently, the pharmacokinetic behavior of the PEGylated NELL-1 was examined in mice.

### 2. Material and Methods

### 2.1 Synthesis of PEGylated NELL-1

PEGylated NELL-1 (NELL-PEG) was synthesized from linear PEG-NHS Mw 5 kDa (PEG 5k, Sigma-Aldrich, USA), linear PEG-NHS Mw 20 kDa (PEG 20k, NOF America corporation, Japan), and 4-arm-branched PEG-NHS Mw 40 kDa (PEG 40k, NOF America corporation, Japan) according to a protocol described previously with modification [18]. Briefly, 0.5 mg of NELL-1 (10.0 mg/mL) was diluted to a concentration of 2.0 mg/mL in PBS buffer (pH6.5, 0.1 M), then added with 10 μl of 62.5 mg/mL PEG 5k, or 40 μl of 62.5 mg/mL PEG 20k, or 80 μl of 62.5 mg/mL PEG 40k at a NELL-1 to PEG molar ratio of 1:100. The PEGylation system was reacted under 300 rpm magnetic stirring for 12 h at 4° C. The obtained NELL-PEG was purified by loading the reaction mixture onto a Sephadex G-25M column (Sigma-Aldrich, USA), eluting the column with 3.0 mL of PBS solution (1×, pH 7.4), and collecting the fractions (0.25 mL/fraction) that mainly consisted of NELL-PEG determined by GPC method, then a 24 h dialysis against distilled water was performed using suitable dialysis cassettes (Fisher, USA) to remove any unreacted PEG molecules.

### 2.2 GPC Characterization of NELL-PEG

The synthesis of PEGylated NELL-1 was confirmed by the gel permeation chromatography (GPC), and naked NELL-1 was used as control in the GPC analysis. Briefly, an ultra-hydrogel linear column (7.8 mm×300 mm, 10 μm grain diameter) was attached to a GPC system (Waters Corp.). The isocratic GPC investigations were performed using PBS buffer (50 mM, added with 0.15 M NaCl, pH 7.5) as mobile phase in a flow rate of 1.0 mL/min. Ten μl of the protein solutions were injected and the GPC curve of the protein was recorded with a UV detector at 280 nm.

### 2.3 Fluorometric Assay of PEG Degree

The degree of PEG modification of NELL-PEG was determined by fluorometric assay with fluorescamine. Briefly, a fresh working solution of fluorescamine in DMSO at a concentration of 1.0 mg/mL was prepared first, and then a 12 μl of fluorescamine solution was added and mixed to 36 μl of NELL-PEG or naked NELL-1 in 0.1 M phosphate buffer (pH 8.0) in a 96-well clear bottom black micro-plate. A series of samples with different protein concentration was prepared and reacted for 15 min at 25° C. under gentle shaking. Then the fluorescence intensity was determined using a plate reader (Infinite F200, Tecan Group Ltd.) at an excitation wavelength of 390 nm and an emission wavelength of 475 nm. The naked NELL-1 was used as a control to determine the unreacted amine groups of the NELL-PEG.

### 2.4 Thermal Shift Assay

The thermal stability of NELL-PEG was evaluated by thermal shift assay method using a 7300 real-time PCR system (Applied Biosystems, CA). Prior to use, the environmentally sensitive fluorescent dye SYPRO Orange stock solution in DMSO (5,000×, Sigma) was diluted 1:125 in PBS. The samples were prepared in a 96-well plate in triplicate containing 3 μl of NELL-PEG (1.0 mg/mL), 2.5 lil of freshly diluted SYPRO orange (40×) and 19.5 μl of PBS buffer (0.01 M, pH 7.4), then the plate was sealed with optical quality sealing tape and centrifuged at 4,000 rpm for 2 min. The fluorescent intensity was monitored as the plate was heated from 298 to 368 K in an increment of 1 K/min. The fluorescent data was analyzed using a Boltzmann model and the melting point (Tₘ) was calculated.

### 2.5 Cytotoxicity Assay of NELL-PEG

The cytotoxic effect of PEGylated NELL-1 and naked NELL-1 was tested by Alamar blue assay. MC3T3 cells were seeded in 96-well cell culture plates at a density of 10,000 cells/well and kept overnight in growth medium at 37° C. in a 5% CO₂ incubator. Then 100 μl of different concentration of NELL-PEG and naked NELL-1 medium were added into the cells and incubated for 24 h at 37° C. MC3T3 cells without NELL-1 protein were used as negative control for 100% cell viability. Then the medium was replaced by growth medium containing 10% Alamar blue, and incubated for 1 h at 37° C. in dark. The fluorescent signal was measured by a plate reader (Infinite F200, Tecan Group Ltd.) with 560 nm excitation wavelength and 595 nm emission wavelength. The results were calculated by averaging the values obtained and subtracting the average value of no-cell control. The cytotoxicity of each NELL-PEG at each concentration was estimated in triplicate and analyzed statistically using one-way ANOVA for multiple comparisons.

### 2.6 Cells

### 2.6.1 Mouse Calvarial Osteoblast Cells

The mouse calvarial osteoblast cells were isolated from calvaria (frontal and parietal bones) of 3-5 day old mice (strain C57BL/6). Briefly, the calvaria were harvested aseptically and the periosteal layers on both sides were carefully stripped off with blade under sterile PBS solution, then rinsed and cut into trivial bone blocks using scissors. The bone blocks were immersed into 5 mL of trypsin-collagenase solution (DMEM containing 0.1% collagenase type I and 0.125% trypsin), incubated under a 150 rpm shaker at 37° C. for 15 min. Then the supernatant containing cells was transferred into a 50 mL centrifuge tube and spun down the cells at 1,500 rpm for 5 min. The supernatant was discarded and the cells were suspended in growth medium (DMEM containing 10% FBS, 50 U/mL penicillin and 50 ng/mL streptomycin). The cell extraction process was repeated 5 times. The obtained cells were pooled and cultured in growth medium at 37° C. incubator with 5% CO2. After reaching 80% confluence, cells were split and passage 2 was used to conduct in vitro bioactivity experiment.

2.6.2 hPSC Cells

The human perivascular stem cells (hPSC) were isolated from fresh adipose tissues as described before [19]. The cells were cultured in growth medium (DMEM containing 20% FBS, 50 U/mL penicillin and 50 ng/mL streptomycin) in a sterile incubator at 37° C. and 5% CO2. Once reached 80% confluence, the cells were passaged and passage 2 was used to conduct in vitro bioactivity experiment.

### 2.7 Bioactivity In Vitro

### 2.7.1 ALP Testing

The bioactivity of the PEGylated NELL-1 was determined by measuring its ability to increase the expression of alkaline phosphatase (ALP) in the mouse calvarial osteoblast cells. The cells were cultured for 9 days in osteogenic medium at 37° C. in a 5% CO₂ humidified incubators, then the cells were solubilized in 200 (4.1 lysis buffer (0.2% NP40 plus 1.0 mM magnesium chloride) for 15 min at 4° C. The cell lysate was scrapped and centrifuged for 5 min at 12,000 rpm, then 15 μl of cell supernatant was mixed with 200 μl of ALP substrate buffer consisting of 0.4 mg p-nitrophenol phosphate, 100 μl alkaline buffer A (A9226, sigma) and 100 μl distilled water. After incubated for 15 min at 37° C., 30 μl of 1.0 N NaOH was added to stop the reaction, then followed by colorimetric detection at 405 nm. Protein amount in the corresponding well was determined by Bradford assay. The quantitative analysis of ALP activity was normalized by the OD405 values of the ALP data to the corresponding protein amount. The measurements were performed in triplicate for each sample.

### 2.7.2 Mineralization Testing

The mineral formation of calcium phosphate of the hPSC cells in osteogenic medium was assessed using Alizarin Red S (ARS) staining as described in a previous paper [20]. Briefly, the hPSC cells were incubated on a 24-well plate in osteogenic medium consisting of α-MEM, 10% FBS and mineralization-inducing components including L-ascorbic acid (50 mg/mL, Sigma, US) and β-glycerophosphate disodium salt (10 mM, MP Biomedicals, US). The medium was changed every 3 days. After 15-day incubation, the cell monolayers were washed with 2 mL PBS (1×, pH 7.4) per well, then fixed with 1 mL of 10% formaldehyde for 15 min. Next, the fixed cells were washed 3 times with distilled water and stained with ARS solution (40 mM, pH 4.2) for 20 min, then excess dye was removed by washing 4 times with distilled water, and the wells with stained mineral nodules were imaged by digital camera. Then the ARS in cells was leached by heating to 85° C. for 10 min in 10% acetic acid. The cell lysate was centrifuged at 14,000 rpm for 10 min, then the supernatant was collected and adjusted to pH 4.1-4.5 with 10% ammonium hydroxide. The amount of ARS in each well was quantified at 450 nm. The experimental conditions were conducted in quadruplicate.

### 2.8 In Vivo Study

### 2.8.1 Preparation of FITC-Labeled NELL-1 and NELL-PEG

The NELL-1 and PEGylated NELL-1 were labeled with fluorescein isothiocyanate (FITC) for in vivo study. Briefly, 50 [ig of FITC (Sigma) was added into 0.25 mL of NELL-1 (4.0 mg/mL) in a 0.1 M sodium carbonate-bicarbonate buffer (pH 9.0) at a 50:1 molar ratio of FITC to protein, and reacted for 3 h under magnetic stirring at 250 rpm at room temperature. The FITC tagged NELL-1 was separated from unreacted FITC by passing through a Sephadex G-25 column, and the fractions containing FITC-NELL-1 were collected and pooled. The concentration and the degree of labeling (fluorescein/protein ratio, F/P) of the conjugate was determined by measuring its absorbance at 280 nm and 495 nm using a spectrophotometer. The vial of labeled protein was wrapped with aluminum foil to protect from light and stored at −20° C. The FITC labeled NELL-PEGs were prepared with the same procedure as above.

### 2.8.2 Animal and Pharmacokinetic Study

The 3-month-old female mice (CD-1 strain, Charles River Laboratories) were used to determine the residence of PEGylated NELL-1 in vivo. They were housed under laboratory conditions, and the experiment protocols for animal studies were approved by the UCLA Chancellor's Animal Research Committee.

The mice were randomly divided into 4 groups, 6 mice for each group. Group 1,2,3,4 was injected with FITC-NELL-1, FITC-NELL-PEG-5k, FITC-NELL-PEG-20k and FITC-NELL-PEG-40k, respectively. Each mouse was administered with 100 μl single IV bolus dose of protein (1.25 mg protein/kg of mouse body weight) from the lateral tail vein. The blood samples were drawn from the mouse retro-orbital sinus with a capillary tube at 0.5 and 24 h, then transferred into serum separator tubes. The serum was separated by centrifugation at 10,000 rpm for 5 min. The concentration of NELL-1 or NELL-PEG in serum was analyzed by monitoring the fluorescence intensity of FITC using a plate reader (Infinite F200, Tecan Group Ltd.). The blood tubes were wrapped with aluminum foil to protect from light during sample process.

### 2.9 Statistical Analyses

Statistical analyses were performed using one-way ANOVA for multiple comparisons and Student's t-test for two-group comparisons at 95% confidence levels. Data are expressed as means±SD, and value of (*) P<0.05 was considered statistically significant differences, (**) P<0.001 was considered statistically highly significant differences.

### 3. Results

### 3.1 Successful Synthesis of PEGylated NELL-1

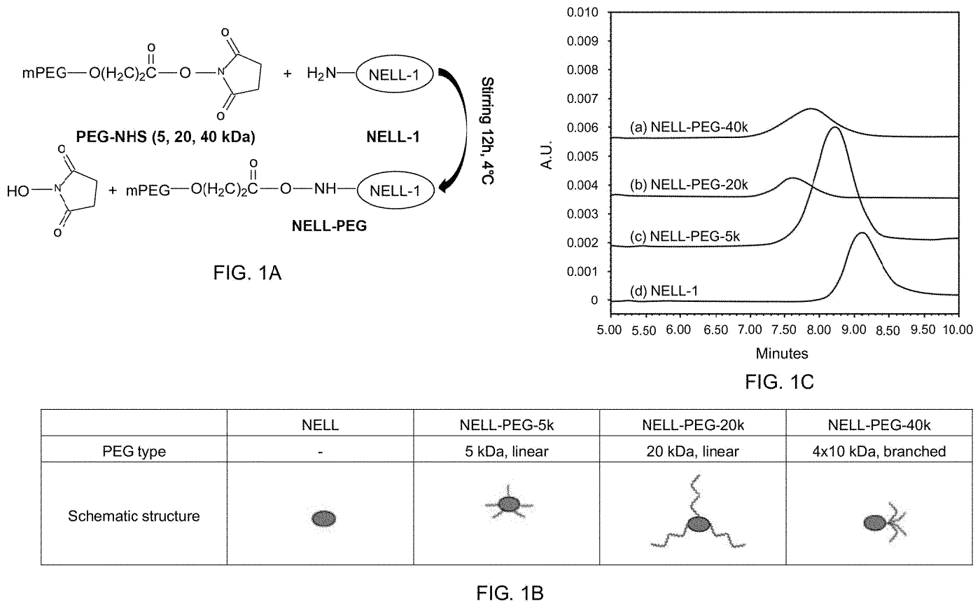

Three sizes of PEG were used in the experiment: the linear mPEG-NHS Mw 5 kDa, linear mPEG-NHS Mw 20 kDa, and 4-arm-branched mPEG-NHS Mw 40 kDa. During conjugation, the N-hydroxysuccinimide group of PEG-NHS was covalently reacted to the ε-amine group presented on lysine side chains or the α-amine group of the NELL-1 protein. The reaction mechanism was illustrated in FIG. 1a. The schematic structures of the three PEGylated NELL-1 with different PEG molecules were shown in FIG. 1b. The PEGylation reaction was successfully confirmed by GPC characterization (FIG. 1c). The GPC system with a size exclusion column can separate different proteins on a basis of size. The NELL-PEG with larger size came out earlier and possessed a shorter retention time (RT). FIG. 1c showed the elution curves of the three NELL-PEG conjugates. The RT of NELL-1, NELL-PEG-5k, NELL-PEG-20k and NELL-PEG-40k were 8.614, 8.221, 7.632 and 7.876 min, respectively. The RT of PEGylated NELL-1 was smaller than that of the naked NELL-1, which indicated that the PEG molecules were successfully linked to NELL-1 and led to an increased size. The symmetrical peak shape of the NELL-PEG and no PEG peak observed in GPC profiles suggested the purification process was effective in the production. Therefore, the GPC characterization indicates that the chemical synthesis of NELL-PEG was successful.

### 3.2 PEG Modification Degree of PEGylated NELL-1

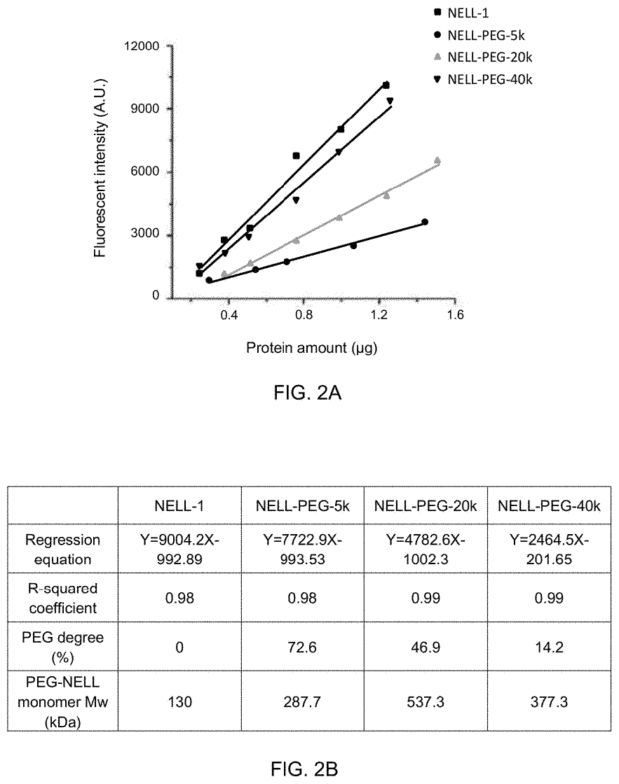

The PEGylation degree of the NELL-PEG was determined by the fluorescamine method as described above. The plots were shown in FIG. 2a. The regression coefficients for all the groups were close to 1. The amount of the residual amine group and PEG modification degree of each NELL-PEG were calculated based on the slopes of the linear regression analysis, and the results were listed in FIG. 2b. It can be seen that the PEG 10 degrees of the NELL-PEG-5k, NELL-PEG-20k, and NELL-PEG-40k were 72.6%, 46.9% and 14.2%, respectively. The results showed that the PEG degree of NELL-PEG decreased as the molecular weight of PEG increased. This is because the NHS group in linear PEG with smaller Mw will more favorably react with the amine group in NELL-1 than larger Mw PEG, especially for the PEG with branched structure. The average molecular weights of the PEGylated proteins were estimated by the data of PEG modification degree. The results indicated that the NELL-PEG-20k monomer possessed the highest molecular weight among the three conjugates. The Mw of NELL-PEG-20k was about 4.1 times of naked NELL-1, 1.9 times of NELL-PEG-5k, and 1.4 times of NELL-PEG-40k.

### 3.3 PEGylation of NELL-1 Increases its Thermal Stability

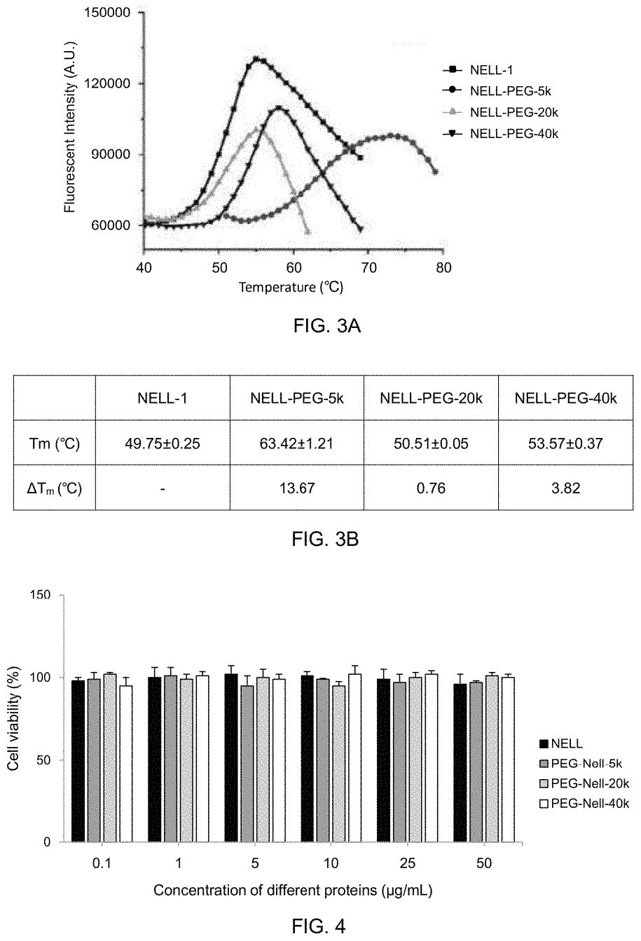

The thermal stability of the PEGylated NELL-1 was investigated by the thermal shift assay. The fluorescent curves of different NELL-PEG and naked NELL-1 were shown in FIG. 3a. The Tₘ of the proteins analyzed by a Boltzmann model and the corresponding thermal shift amount (ΔTₘ=Tₘ−T₀) was listed in FIG. 3b. The Tₘ of naked NELL-1 was 49.75° C. After PEGylation, the Tₘ was increased to 63.42, 50.51, 53.57° C. for NELL-PEG-5k, NELL-PEG-20k, and NELL-PEG-40k, respectively. The protein melting point of the NELL-1 was shifted to higher temperature after PEGylation, which suggested the stability of the NELL-1 protein was enhanced. Interestingly, the thermal stability of NELL-PEG-5k was much higher than the NELL-PEG-20k and NELL-PEG-40k. This can be explained by the difference in PEG modification degrees. A Nell-1 molecule linked with more PEG molecules can lead to higher stability. The PEG degree assay confirmed that each NELL-PEG-5k molecule possessed 31.5 PEG molecules, which was much higher than the 20.4 PEG molecules of NELL-PEG-20k and 6.2 PEG molecules of NELL-PEG-40k.

### 3.4 PEGylated NELL-1 is Nontoxic to Osteoblastic Cells

In this study, the effect of PEG type and concentration on NELL-PEG cytotoxicity was investigated in MC3T3 cell line by Alamar blue assay, and the result was shown in FIG. 4. Compared to the control group, the PEGylated NELL-1 and naked NELL-1 tested at concentrations up to 50 μg/mL did not show appreciable cytotoxicity to MC3T3 cells. After analysis using one-way ANOVA by SPSS, there was no significant difference between different NELL-PEG groups. The results indicate that the treatment of all the PEGylated NELL-1 at all concentrations is nontoxic to MC3T3 cells. In addition, no cytotoxic effect of NELL-PEG on primary hPSCs and mouse calvarial cells was observed in the following osteoblastic differentiation experiments.

### 3.5 PEGylated NELL-1 Preserves Pro-Osteoblastic Bioactivity In Vitro

### 3.5.1 ALP of Mouse Osteoblast Cells

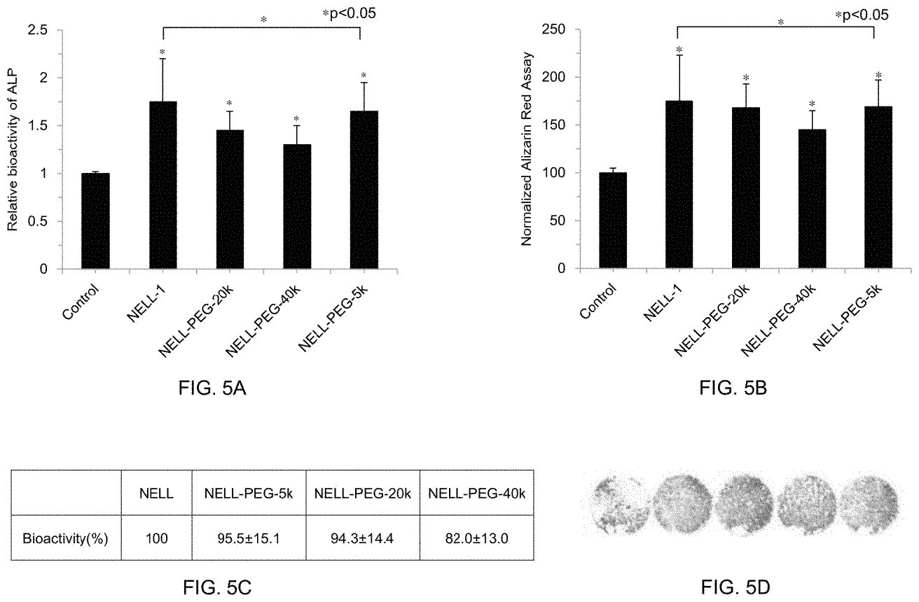

To examine the bioactivity of the NELL-PEG conjugates, their ability to facilitate the differentiation of the mouse osteoblast cells in vitro was investigated by ALP testing. The mouse osteoblast cells were incubated for a 9-day exposure to NELL-1 or NELL-PEGs in osteogenic medium, then the ALP activity of the cells was normalized on the basis of protein content in each well. The relative bioactivity of the NELL-PEG was shown in FIG. 5a. The result showed that three kinds of NELL-PEG did not show significant difference after analysis by one-way ANOVA with multiple comparisons, but their bioactivity was significantly higher than the control group without NELL-1 (*p<0.05). Therefore, the PEGylated NELL-1 significantly enhanced the ALP activity of mouse osteoblast cells compared to control group.

3.5.2 Mineralization of hPSC

Subsequently, the ability of the NELL-PEG to facilitate the hPSC mineralization in vitro was investigated. After 15-day incubation in osteogenic medium, the hPSC cells were stained with ARS solution (FIG. 5d) depicted the images of the whole wells containing stained calcium mineralization, which showed that all the protein groups (naked NELL-1, NELL-PEG-5k, NELL-PEG-20k, and NELL-PEG-40k) significantly increased the matrix mineralization compared to the negative control group (NELL-1 free). The ARS in the wells was next extracted from the hPSC cell monolayer by acetic acid for bioactivity quantification (FIG. 5b). The result showed that the bioactivity of the NELL-PEG measured by mineralization testing with hPSC cells was similar to the ALP testing with mouse osteoblast cells. The bioactivity of the three kinds of NELL-PEG was significantly higher than the NELL-1 free control group (*p<0.05). The relative activity of different PEGylated NELL-1 protein compared to naked NELL-1 was listed in FIG. 5c. The NELL-PEG exhibited slightly decreased bioactivity compared to naked NELL-1, although they did not show significant difference after statistical analysis by SPSS. The decreased bioactivity of the NELL-PEG groups (especially for the NELL-PEG-40k) suggested that the PEGylation affected the bioactivity of NELL-1. Because more protein surface can be covered by branched PEG, the 4-arm branched PEG-40k could provide better protection for NELL-1 against attack by enzymes and other proteins in vivo [21], but it also hindered the interaction between cells and NELL-1 molecules, thus leading to the reduced bioactivity. The experiment shows that the bioactivity of the PEGylated NELL-1 was preserved and still can facilitate the osteogenesis of hPSC cells.

### 3.6 NELL-1 PEGylation Significantly Extends its Circulation Time in Mice

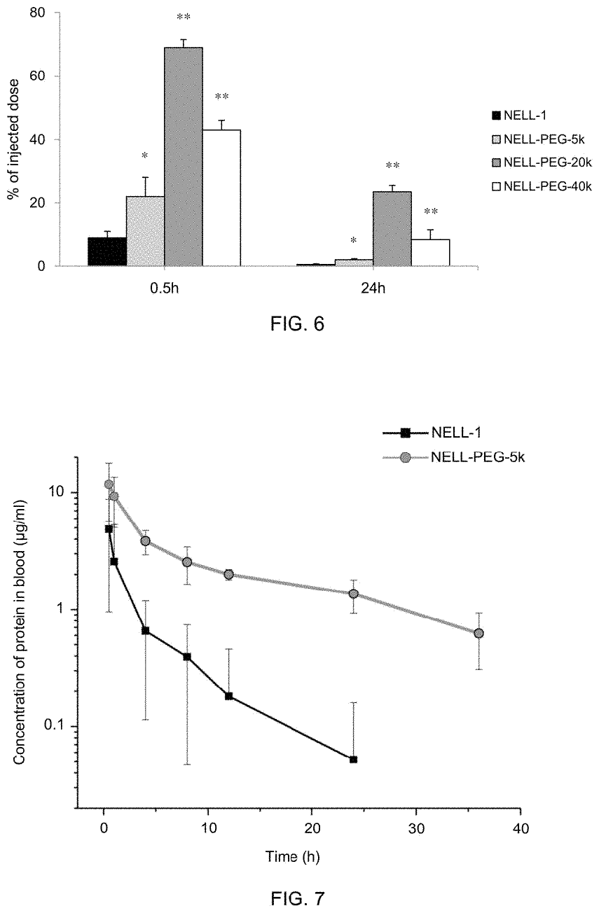

The PEGylated NELL-1 and naked NELL-1 were labeled with FITC and used for monitoring their concentrations in blood. The F/P ratio of the obtained FITC-protein was 4.36 after analysis. In order to check whether the PEGylation of NELL-1 can increase its circulation time, the pharmacokinetics of the naked NELL-1 and PEGylated NELL-1 in CD-1 mice were investigated. FIG. 6 showed the remaining amount (%) of NELL-1 and NELL-PEG in mice at different time points after a single intravenous injection. For the naked NELL-1, only 9.3±3.7% of the initial dose was detected in mouse blood at 0.5 h, but for the PEGylated NELL-1, the protein amounts at 0.5 h were significantly higher (NELL-PEG-5k, NELL-PEG-20k, NELL-PEG-40k were 22.6±8.3%, 67.1±3.9%, 44.0±4.3%, respectively). After 24 h administration, the amounts of PEGylated NELL-1 in vivo remained significantly higher compared to naked NELL-1, which was almost gone from the mice at that time. Furthermore, among the three kinds of NELL-PEG examined, the NELL-PEG-20k with linear 20 kDa PEG had significantly higher remaining amount in blood compared to the linear 5 kDa PEG and the branched 40 kDa PEG both at 0.5 h and 24 h. Therefore, NELL-1 PEGylation can significantly improve circulation time of NELL-1 in vivo.

### 4. Discussion

Osteoporosis is a progressive bone disease with the characteristic of a decrease in bone mass and density. There is greater osteoclast activity than osteoblast activity in patients, thus the net rate of bone resorption exceeds the rate of bone formation [22], NELL-1 can specifically increase osteoblast differentiation and activity, but without a concomitant osteoclast response [7], which make it possible to be a promising therapy for osteoporosis treatment. Furthermore, unlike BMP-2, NELL-1 is a downstream mediator of runt-related transcription factor 2 (Runx2) during bone formation, thus less side effects occurred during application in vivo [1]. As a potent growth factor, NELL-1 has been studied preclinically for the induction of bone formation [1]. Because of the undesirable side effects of BMP-2 including excessive and ectopic bone formation, bone resorption, etc. [23, 24], NELL-1 protein has the potential of replacing BMP-2 in clinical application. Similar to other native protein drugs, NELL-1 has a relatively short half-life time in vivo after intravenous injection due to rapid clearance from the body, which limits its application as a therapy for osteoporosis. Meanwhile, the systemic nature of osteoporosis has called for intravenous medication administration as a therapeutic remedy. Therefore, in the present study, the circulation time of NELL-1 was extended by PEGylation in order to meet the demand of the osteoporosis treatment. PEGylation technology can improve the elimination half-life time of native proteins by preventing their renal clearance and decreasing protease degradation in vivo [25].

In this study, the effect of PEGylation on the circulation time of NELL-1 in mice was examined in order to determine whether it can improve the drug delivery in vivo or not. We first synthesized NELL-1 with three different PEG sizes (5, 20, and 40 kDa), and investigated their physical properties with GPC, fluorometric assay and thermal shift assay, then evaluated their cytotoxicity and bioactivity in vitro and pharmacokinetics in vivo.

GPC analysis of the PEGylated NELL-1 was conducted in order to monitor the synthesis and characterize the obtained conjugates. The decreased retention time of the PEGylated NELL-1 compared to naked NELL-1 indicated that they were successfully obtained by the adopted PEGylation method. Interestingly, the retention time of NELL-PEG-40k with branched PEG molecule was smaller than that of NELL-PEG-20k with linear PEG, indicating that the size of the NELL-PEG-40k was hydrodynamically smaller than the NELL-PEG-20k. The fluorometric assay data shown in FIG. 2b confirmed that the molecular weight of the NELL-PEG-40k monomer was smaller than NELL-PEG-20k. In addition, It is reported that the conjugated PEG moiety in PEGylated protein is still highly extended, similar to the random-coiled unconjugated PEG [26, 27], therefore, the NELL-PEG is likely to assume a highly elongated conformation, as depicted in FIG. 1b. Each chain of the 4 arm-branched PEG-40k actually is 10 kDa, which is shorter than the linear PEG-20k when they are in the highly extended conformation. This also explains why the hydrodynamic size of NELL-PEG-40k is smaller than NELL-PEG-20k.

The selected PEGylation method in current research is a random reaction, by which a PEG molecule can be coupled to all the accessible secondary NH₂ groups of lysine located at the surface of NELL-1 protein, which implies that one or multiple PEG molecules can be linked to each NELL-1 molecule. Therefore, it is necessary to quantify the number of PEG molecules coupled to NELL-1. The result of the fluorometric assay showed that there were 43.4 lysine residues accessible for chemical modification in each NELL-1 monomer. After PEGylation with different PEG molecules, the PEG modification degrees of NELL-PEG-5k, NELL-PEG-20k and NELL-PEG-40k were 72.6%, 46.9% and 14.2%, respectively. Thus there are 31.5, 20.4 and 6.2 PEG molecules conjugated to each NELL-1 molecule on average for NELL-PEG-5k, NELL-PEG-20k, and NELL-PEG-40k, respectively.

The intrinsic thermodynamic stability of the naked NELL-1 and PEGylated NELL-1 was determined by the thermal shift assay. Basically, PEGylated NELL-1 has a higher Tₘ than the naked NELL-1 as shown in the FIG. 3. Therefore, covalently linked PEG moiety can interact and stabilize the conformation of the NELL-1 in the immediate microenvironment. Interestingly, the NELL-PEG-20k, with the highest Mw, did not possess the highest thermal stability, which indicates that the thermal stability is independent of the molecular weight of the PEG moiety. Plesner et al. [28] have investigated the effect of PEG size on the stability of PEG-BSA, and concluded that the larger size of PEG chain did not contribute more to the stability of protein, which is supported by our experiment. Although it is reported that the PEGylation effect on the thermal stability of protein depends on several factors including the PEG degree, the site of the polymer conjugation, and the specific local interaction between the PEG and protein surface [29, 30], it is difficult to predict the thermal stability of PEGylated protein, because PEGylation may increase the stability for some proteins [31-33], but it also could decrease the stability for other proteins [28, 34].

The PEG related toxicity is rare for PEGylated proteins based on previous studies [35-37]. In the current research, the PEGylated NELL-1 with different PEG structures did not show any toxicity on MC3T3 cells at various concentrations, which indicate that the NELL-PEG are safe for the biological experiment.

Subsequently, the bioactivity of NELL-PEG was assessed by a cell-based assay. PEGylation modification usually led to reduced protein bioactivity because of the PEG resistance between protein and targeted receptors [25]. In the current study, two kinds of primary cells, the hPSC cells and mouse osteoblast cells, were used to determine the pro-osteoblastic bioactivity of naked and PEGylated NELL-1. Similar results were obtained by the mineralization testing of hPSC cells and the ALP activity testing of mouse osteoblast cells. All the PEGylated Nell-1 can significantly facilitate the differentiation of hPSC and mouse osteoblast cells compared to control group (NELL-1 free). Although PEGylated NELL-1 showed a reduced biological activity (especially for the NELL-PEG-40k) compared to naked NELL-1, the data did not show a statistically significant difference after analysis. Therefore, the bioactivity of NELL-1 was preserved after PEGylation.

PEGylation was used to increase NELL-1's residence in circulation system in the current research, so the assessment of the pharmacokinetics of NELL-1 after PEGylation is vital for its efficiency over naked NELL-1. In the current study, the remaining amounts of NELL-PEG and NELL-1 in mice after a single intravenous bolus injection were examined. The study demonstrated that NELL-PEG had a significantly higher remaining amount compared to naked NELL-1 in vivo at 0.5 h and 24 h. The result indicated that PEGylated NELL-1 could reside in mice for a much longer time than naked NELL-1 under equal initial dose. There was almost no naked NELL-1 that could be detected in blood at 24 h, while the PEGylated NELL-1 still could be tracked at that time, especially for the NELL-PEG-20k, for which the remaining amount was still up to 24%. These data confirm that PEGylated NELL-1 can lead to a much longer circulation time in vivo. Of the various sized PEGylated NELL-1 molecules, the remaining amount and circulation time of NELL-PEG-40k were smaller than the NELL-PEG-20k in blood. The reason is that both Mw and hydrodynamic size of NELL-PEG-40k are smaller than the NELL-PEG-20k as confirmed by the fluorometric assay and GPC characterization.

### 5. Conclusion

In the present study, we have prepared three kinds of NELL-PEG conjugates that varied in the size and structure of PEG molecules: linear 5 KDa, linear 20 kDa and branched 40 kDa, and characterized their properties by in vitro and in vivo testings. Compared to naked NELL-1, all three NELL-PEG conjugates showed not only a preserved bioactive potency in vitro, but also a significantly improved circulation time in vivo observed in mice. Further studies are needed to evaluate the effect of PEGylated NELL-1 on the treatment of osteoporosis with animal models, and the long-circulating NELL-PEG conjugates with a maintained osteogenic activity represent a solid step toward to developing an effective osteoporosis therapy.

### REFERENCES

[1] Zhang X, Zara J N, Siu R K, Ting K, Soo C. The Role of NELL-1, a Growth Factor Associated with Craniosynostosis, in Promoting Bone Regeneration. J Dent Res 2010; 89:865-78.
[2] Aghaloo T, Cowan C M, Chou Y F, Zhang X, Lee H F, Miao S, et al. Nell-1-induced bone regeneration in calvarial defects. Am J Pathol 2006; 169:903-15.
[3] Lu S S, Zhang X, Soo C, Hsu T, Napoli A, Aghaloo T, et al. The osteoinductive properties of Nell-1 in a rat spinal fusion model. Spine J 2007; 7:50-60.
[4] Siu R K, Zara J N, Hou Y P, James A W, Kwak J, Zhang X, et al. NELL-1 Promotes Cartilage Regeneration in an In Vivo Rabbit Model. Tissue Eng Part A 2012; 18:252-61.
[5] Kuroda S, Oyasu M, Kawakami M, Kanayama N, Tanizawa K, Saito N, et al. Biochemical characterization and expression analysis of neural thrombospondin-1-like proteins NELLI and NELL2. Biochem Biophys Res Commun 1999; 265:79-86.
[6] Siu R K, Lu S S, Li W, Whang J, McNeill G, Zhang X, et al. Nell-1 Protein Promotes Bone Formation in a Sheep Spinal Fusion Model. Tissue Eng Part A 2011; 17:1123-35.
[7] Kwak J, Zara J N, Chiang M, Ngo R, Shen J, James A W, et al. NELL-1 Injection Maintains Long-Bone Quantity and Quality in an Ovariectomy-Induced Osteoporotic Senile Rat Model. Tissue Eng Part A 2013; 19:426-36.
[8] Karasik D, Hsu Y H, Zhou Y H, Cupples L A, Kiel D P, Demissie S. Genome-Wide Pleiotropy of Osteoporosis-Related Phenotypes: The Framingham Study. J Bone Miner Res 2010; 25:1555-63.
[9] Zhang X, Ting K, Pathmanathan D, Ko T, Chen W W, Chen F, et al. Calvarial Cleidocraniodysplasia-Like Defects With ENU-InducedNell-1 Deficiency. J Craniofac Surg 2012; 23:61-6.
[10] Li W, Lee M, Whang J, Siu R K, Zhang X, Liu C, et al. Delivery of lyophilized Nell-1 in a rat spinal fusion model. Tissue Eng Part A 2010; 16:2861-70.
[11] Harris J M, Chess R B. Effect of pegylation on pharmaceuticals. Nat Rev Drug Discovery 2003; 2:214-21.
[12] Morpurgo M, Veronese F M. Conjugates of peptides and proteins to polyethylene glycols. Bioconjugation Protocols: Springer; 2004. p. 45-70.
[13] Pasut G, Guiotto A, Veronese FM. Protein, peptide and non-peptide drug PEGylation for therapeutic application. Expert Opin Ther Pat 2004; 14:859-94.
[14] Wang J, Hu T, Liu Y, Zhang G, Ma G, Su Z. Kinetic and stoichiometric analysis of the modification process for N-terminal PEGylation of staphylokinase. Anal Biochem 2011; 412:114-6.
[15] Arakawa T, Prestrelski S J, Narhi L O, Boone T C, Kenney W C. Cysteine 17 of recombinant human granulocyte-colony stimulating factor is partially solvent-exposed. J Protein Chem 1993; 12:525-31.
[16] Veronese F M, Pasut G. PEGylation, successful approach to drug delivery. Drug Discov Today 2005; 10:1451-8.
[17] Nojima Y, Iguchi K, Suzuki Y, Sato A. The pH-dependent formation of PEGylated bovine lactoferrin by branched polyethylene glycol (PEG)-N-hydroxysuccinimide (NHS) active esters. Biol Pharm Bull 2009; 32:523-6.
[18] Bansal R, Post E, Proost J H, de Jager-Krikken A, Poelstra K, Prakash J. PEGylation improves pharmacokinetic profile, liver uptake and efficacy of Interferon gamma in liver fibrosis. J Controlled Release 2011; 154:233-40.
[19] James A W, Zara J N, Zhang X, Askarinam A, Goyal R, Chiang M, et al. Perivascular stem cells: a prospectively purified mesenchymal stem cell population for bone tissue engineering. Stem Cells Transl Med 2012; 1:510-9.
[20] Chen F, Walder B, James A W, Soofer D E, Soo C, Ting K, et al. NELL-1-dependent mineralisation of Saos-2 human osteosarcoma cells is mediated via c-Jun N-terminal kinase pathway activation. Int Orthop 2012; 36:2181-7.
[21] Monfardini C, Schiavon O, Caliceti P, Morpurgo M, Harris J M, Veronese F M. Abranched monomethoxypoly (ethylene glycol) for protein modification. Bioconjugate Chem 1995; 6:62-9.
[22] Klibanski A, Adams-Campbell L, Bassford T, Blair S N, Boden S D, Dickersin K, et al. Osteoporosis prevention, diagnosis, and therapy. J Am Med Assoc 2001; 285:785-95.
[23] Shields L B E, Raque G H, Glassman S D, Campbell M, Vitaz T, Harpring J, et al. Adverse effects associated with high-dose recombinant human bone morphogenetic protein-2 use in anterior cervical spine fusion. Spine 2006; 31:542-7.
[24] Benglis D, Wang M Y, Levi A D. A comprehensive review of the safety profile of bone morphogenetic protein in spine surgery. Neurosurgery 2008; 62:ONS423-ONS31.
[25] Jevsevar S, Kunstelj M, Porekar V G. PEGylation of therapeutic proteins. Biotechnol J 2009; 5:113-28.
[26] Fee C J. Size comparison between proteins PEGylated with branched and linear poly (ethylene glycol) molecules. Biotechnol Bioeng 2007; 98:725-31.
[27] Caliceti P, Veronese F M. Pharmacokinetic and biodistribution properties of poly (ethylene glycol)-protein conjugates. Adv Drug Deliv Rev 2003; 55:1261-77.
[28] Plesner B, Fee C J, Westh P, Nielsen A D. Effects of PEG size on structure, function and stability of PEGylated BSA. Eur J Pharm Biopharm 2011; 79:399-405.
[29] Gokam Y R. Hydrodynamic behavior and thermal stability of a PEGylated protein: Studies with hen egg lysozyme: University of New Hampshire (Department of Biochemistry and Molecular Biology); 2003.
[30] Pandey B K, Smith M S, Torgerson C, Lawrence P B, Matthews S S, Watkins E, et al. Impact of Site-Specific PEGylation on the Conformational Stability and Folding Rate of the Pin WW Domain Depends Strongly on PEG Oligomer Length. Bioconjugate Chem 2013; 24:796-802.
[31] Yang C, Lu D, Liu Z. How PEGylation enhances the stability and potency of insulin: a molecular dynamics simulation. Biochemistry 2011; 50:2585-93.
[32] Meng W, Guo X, Qin M, Pan H, Cao Y, Wang W. Mechanistic Insights into the Stabilization of srcSH3 by PEGylation. Langmuir 2012; 28:16133-40.
[33] Chiu K, Agoubi L L, Lee I, Limpar M T, Lowe Jr J W, Goh S L. Effects of polymer molecular weight on the size, activity, and stability of PEG-functionalized trypsin. Biomacromolecules 2010; 11:3688-92.
[34] Garcia-Arellano H, Valderrama B, Saab-Rincón G, Vazquez-Duhalt R. High temperature biocatalysis by chemically modified cytochrome c. Bioconjugate Chem 2002; 13:1336-44.
[35] Webster R, Didier E, Harris P, Siegel N, Stadler J, Tilbury L, et al. PEGylated proteins: evaluation of their safety in the absence of definitive metabolism studies. Drug Metab Dispos 2007; 35:9-16.
[36] Modi M W, Fulton J S, Buckmann D K, Wright T L, Moore D J. Clearance of pegylated (40 kDa) interferon alfa-2a Pegasys is primarily hepatic. Hepatology 2000; 32:371A.
[37] Viau A T, Abuchowski A, Greenspan S, Davis F F. Safety evaluation of free radical scavengers PEG-catalase and PEG-superoxide dismutase. J Free Radic Biol Med 1986; 2:283-8.

### Example 2. Pharmacokinetics and Osteogenic Potential of PEGylated NELL-1 In Vivo after Systemic Administration

### Summary

Osteoporosis is a skeletal disorder attributable to an imbalance in osteoblast and osteoclast activity. NELL-1, a secretory protein that promotes osteogenesis while suppressing osteoclastic activity, holds potential as an osteoporosis therapy. Recently, we demonstrated that PEGylation of NELL-1 significantly improves its thermostability while preserving its bioactivity in vitro. However, the effect of PEGylation on the pharmacokinetics and osteogenic potential of NELL-1 in vivo have yet to be investigated. The present study demonstrated that PEGylation of NELL-1 significantly increases the elimination half-life time of the protein from 5.5 h to 15.5 h while distributing more than 2-3 times the amount of protein to bone tissues (femur, tibia, vertebrae, calvaria) in vivo when compared to naked NELL-1. In addition, microCT and DXA analyses demonstrated that systemic NELL-PEG therapy administered every 4 or 7 days significantly increases not only femoral and lumbar BMD and percent bone volume, but also new bone formation throughout the overall skeleton after four weeks of treatment. Furthermore, immunohistochemistry revealed increased osteocalcin expression, while TRAP staining 15 showed reduced osteoclast numbers in NELL-PEG groups. Our findings suggest that the PEGylation technique presents a viable and promising approach to further develop NELL-1 into an effective systemic therapeutic for the treatment of osteoporosis.

### 1. Introduction

Osteoporosis, the most common metabolic bone disease, affects over 200 million 20 people worldwide with 10 million people affected in the United States alone [1-6]. Therapeutic approaches to osteoporotic bone loss have focused thus far on either anabolic or antiresorptive agents [7, 8] with only one anabolic agent, parathyroid hormone (PTH), approved by the Food and Drug Administration (FDA) for the temporary treatment of osteoporosis. To address the pressing need for new therapies that are both anabolic and anti-osteoclastic [7, 9-11], promising new agents that increase Wnt/β-catenin activity are in development. Wnt/β-catenin signaling plays a key role in directing stem cell differentiation to osteoblasts and in inhibiting osteoclast activity [12, 13]. In addition, decreased Wnt/β-catenin signaling has been implicated in osteoporosis [14, 15]. However, because recombinant Wnts are difficult to produce and deliver, most approaches to increase Wnt/β-catenin signaling involve the blockade of naturally occurring Wnt antagonists via antibodies (e.g., anti-DKK1 or anti-Sclerostin antibodies) [12, 13] to functionally ‘de-repress’ Wnt signaling.

NEL-like molecule-1 (NELL-1), a unique secretory molecule, was first implicated in bone formation by its overexpression in human craniosynostosis [16]. Specifically, NELL-1 is a 700 kDa protein recognized as a potent pro-osteogenic cytokine and was most often studied for its local bone-forming effects [17-24]. NELL-1 has been reported to induce robust osseous healing of critical-sized rat femoral segmental and calvarial defects [17, 25, 26], and also to promote lumbar spinal fusion in rats, sheep [21-23], and non-human primates. Additionally, NELL-1 has been identified not only to demonstrate anti-osteoclastic effects both in vitro and in vivo [19], but also to suppress adipogenesis [27]. Recently, a genome-wide linkage study identified NELL-1 polymorphisms in patients with reduced bone mineral density [28], thus describing an association between NELL-1 and osteoporosis. In accordance with this, our preliminary studies indicated that NELL-1, like Wnt/β-catenin, also acts as a combined anabolic and anti-osteoclastic agent to protect against osteoporotic bone loss. Not only are Nell-1 haploinsufficient mice more prone to osteoporosis, but also the local intramedullary delivery of NELL-1 reverses osteoporotic bone loss in both small (rat) and large (sheep) animal models [20, 29]. Excitingly, we have recently determined that NELL-1 effects occur in large part via activation of Wnt/β-catenin signaling [19] and that systemically delivered NELL-1 potently reverses ovariectomy (OVX)-induced bone loss in mice; however, a relatively frequent administration schedule was required (q2d; 3-4 doses/week) due to the rapid clearance of the native protein [30]. Resultantly, the short circulation time of NELL-1 in vivo was deemed as one of the main limitations for its practical application as a systemic therapy. Therefore, one of the main purposes of the present study was to improve the pharmacokinetics of NELL-1 by structural modification in order to extend its circulation time in vivo.

One of the most biocompatible technologies to prolong the half-life of a protein is to use water-soluble polyethylene glycol (PEG) polymers as macromolecular carriers. PEGylation, the chemical process of modifying a molecule's physiologic and pharmacokinetic characteristics using PEGs, has demonstrated to be both effective and non-toxic [31-34]. Thus far, the FDA has approved 10 marketed PEGylated therapies [34]. PEGylation of a protein not only prolongs its half-life, but also reduces its injection frequency by consequence of extending circulation time in vivo. Seeking to reduce the injection frequency of NELL-1, we previously used PEGylation technology to enhance the systemic pharmacokinetics of NELL-1 [35]. Three different PEGylated types of NELL-1 (5K-linear, 20K-linear, and 40K-branched) were tested, each demonstrating an increased thermostability and a maintained bioactivity in vitro in mouse osteoblast cells and human adipose-derived perivascular stem cells (hPSCs) compared to naked NELL-1 [35]. Importantly, the levels of PEGylated NELL-1 were found to remain significantly higher compared to naked NELL-1 at 24 hours in vivo. As a result, these findings brought PEGylated NELL-1 to the forefront alongside other combined anabolic and antiresorptive agents currently in development such as anti-DKK1 or anti-Sclerostin, both of which are injected every 4 days [36].

In the current study, we investigated the pharmacokinetics of PEGylated NELL-1 in vivo and its resultant osteogenic effect in mice. Previously, we tested three different PEGylation types of NELL-1 (NELL-PEG-5k, NELL-PEG-20k and NELL-PEG-40k) and determined NELLPEG-5k to be the most optimal type of PEGylation for this in vivo study [35]. Thus, in the present study, we examined the osteogenic potential of systemically administered PEGylated NELL-1 (NELL-PEG-5k) compared to naked NELL-1 and a carrier control. In the following text, NELL-PEG refers to NELL-PEG-5k if not mentioned otherwise. Given that the systemic anti-osteoporosis therapeutics currently in development (e.g., anti-DKK1, anti-Sclerostin antibodies) are administered q4d (2 doses/week) [36], we sought to examine the osteogenic potential of NELL-PEG administered at q4d and q7d. Specifically, we aimed to (1) evaluate the pharmacokinetics of NELL-PEG in comparison to naked NELL-1 and a control group in vivo, and then (2) investigate the systemic osteogenic capacity of NELL-PEG when administered at q4d or q7d in vivo. The significance and novelty of the present study is that, upon success, we could develop an efficacious approach to deliver NELL-1 as a growth factor-based systemic osteogenic therapy.

### 2. Materials and Methods

### Animals

All animals were handled in accordance with the institutional guidelines of the Chancellor's Animal Research Committee (ARC) of the Office for Protection of Research Subjects at the University of California, Los Angeles. Animals were housed in a light- and temperature-controlled environment and given food and water ad libitum.

### 2.1 Pharmacokinetic (PK) Study

### 2.1.1 Preparation of FITC-Labeled Protein

NELL-1 and PEGylated NELL-1 were labeled with fluorescein isothiocyanate (FITC) according to our previous paper [35]. FITC (Sigma Aldrich, MO) and either NELL-PEG or NELL-1 (4 mg/mL) were reacted at a 50:1 molar ratio in a 0.1 M sodium carbonate-bicarbonate buffer (pH 9.0) for 3 h at room temperature under magnetic stirring at 250 rpm. The FITCtagged protein was then separated and purified.

### 2.1.2 Pharmacokinetic Study

3-month-old female CD-1 mice (Charles River Laboratories, MA) were used for the PK study. The mice were randomly divided into 2 groups (n=6/group): a FITC-NELL-1 group and a FITC-NELL-PEG group. The mice in each group were intravenously administered 100 μl of sterile solution via the lateral tail vein at a dose of 1.25 mg protein/kg mouse weight (based on protein content). Blood samples were drawn retro-orbitally and collected in a serum separator tube at a series of time-points post-injection (0.5, 1, 4, 8, 12, 24, 36 h). The serum was then separated and the concentrations of FITC-NELL-1 and FITC-NELL-PEG measured by a plate reader (Infinite F200, Tecan Group Ltd., Switzerland). To be protected from light during the sampling process, the tubes of blood and serum were wrapped with aluminum foil. PK parameters were calculated based on individual animal concentrations using the kinetica program. The data were fitted to a two-compartment model and the pharmacokinetic parameters were calculated from the proposed model.

### 2.1.3 Preparation of VivoTag-Labeled Protein

NELL-1 and NELL-PEG were labeled with VivoTag 680XL (PerkinElmer, MA) for a biodistribution study. 30 μl of VivoTag 680XL (10 mg/mL in DMSO) and either 0.5 mL of NELLI or NELL-PEG (2 mg/mL based on protein content) were reacted in a 50 mM sodium carbonate-bicarbonate buffer (pH 8.5) at room temperature for 3 h under magnetic stirring at 250 rpm. The VivoTag 680XL-tagged NELL-1 was then separated from unreacted VivoTag 680XL by filtration chromatography through a Sephadex G-25 column. Next, the fractions containing VivoTag-NELL-1 or VivoTag-NELL-PEG were collected and pooled. The vial of labeled protein was wrapped with aluminum foil to be protected from light and stored at −80° C. before use.

### 2.1.4 Biodistribution Study

For the protein biodistribution study, 3-month-old female CD-1 mice (Charles River Laboratories, MA) were randomly divided into 3 groups (n=3/group): a NELL-PEG group, a NELL-1 group, and a control group (PEG). The mice in each group were administered with a single, 100 μl IV bolus dose via the lateral tail vein at a dose of 1.25 mg/kg (based on protein content) for NELL-PEG and NELL-1 groups and at a dose of 1.52 mg/kg for the PEG group (equal to the PEG amount in the NELL-PEG dose). At 48 h post-treatment, the mice were sacrificed and the organs (liver, kidney, spleen, heart, lungs, brain, muscle, fat, ovary, calvaria, vertebrae, tibia, and femurs) were harvested and imaged using the IVIS Lumina II optical imaging system (Caliper Life Sciences, MA). Special care was taken during dissection to avoid cross-contamination. The organs were weighed and the data were plotted as fluorescence efficiency per gram of tissue weight.

2.2 Systemic Osteogenicity Study (q4d vs. q7d)

### 2.2.1 NELL-PEG Intravenous Injection in Mice

To investigate the osteogenic capacity of systemically administered NELL-PEG, 3-month old female C57BL/6 mice (n=19, mean body weight 20 g, Jackson Laboratory, ME) were used. The mice were randomly divided into 3 groups: a PBS/PEG control (q4d, n=3) group, a NELLPEG (q4d, n=8) group, and a NELL-PEG (q7d, n=8) group. Then, the mice were intravenously injected with 100 μl of PBS/PEG or NELL-PEG via the lateral tail veins at a q4d or q7d dosing interval over a 4-week experimental period. The type of PEG (linear 5 KDa) for NELL-PEG and the optimal doses for PBS/PEG (1.52 mg/kg) and NELL-PEG (1.25 mg/kg) were determined according to our previous studies [30, 35].

### 2.2.2 In Vivo Bone Densitometry by DXA

To monitor bone mineral density (BMD), dual-energy X-ray absorptiometry (DXA) scans were performed weekly using a Lunar PIXImus II densitometer (GE Lunar, WI). Under isoflurane anesthesia, all animals were positioned prone on the imaging pad with the femurs parallel to the direction of the scan and the knee joints flexed at a right angle. Areal BMD was determined with rectangular regions-of-interest (ROIs) placed on distal femurs and lumbar vertebrae (L6) using image analysis software (version 2.10) provided by the manufacturer.

2.2.3 In Vivo microPET/CT Bone Scan Using [¹⁸F] Fluoride Ion

To monitor the overall bone metabolic activity, [⁸F] fluoride ion bone scanning was performed weekly using micro positron emission tomography (microPET) and correlated with anatomical imaging using micro-computed tomography (microCT) at the UCLA Crump Institute for Molecular Imaging. [¹⁸F] localization in the skeleton is dependent on regional blood flow, as well as on new bone formation. [¹⁸F] is substituted for hydroxyl groups in hydroxyapatite and covalently bonds to the surface of new bone; thus, uptake is higher in new bone (osteoid) because of the greater availability of binding sites. In brief, [¹⁸F] fluoride ion was produced at specific activities of approximately 1,000 Ci/mmol using ¹⁸O-labeled water and proton bombardment with a RDS cyclotron (Siemens Medical Solutions USA, Inc., TN). Mice were injected with [¹⁸F] fluoride ion (less than 200 μCi) via the lateral tail vein and kept anesthetized with isoflurane during radioactive probe uptake and clearance for 1 hour, followed by microPET (FOCUS 220 system; Siemens Medical Solutions USA, Inc., TN) and microCT (microCAT II; Siemens Medical Solutions USA, Inc., TN) combination scans lasting 20 minutes. All animals were imaged within ARC-approved rodent imaging chambers to minimize positioning errors during co-registration between microPET and microCT images. MicroPET images were reconstructed using a filtered back projection (FBP) and an iterative three-dimensional maximum a posteriori (MAP) reconstruction algorithm. To ensure the proper anatomical location of ROIs, microPET images were co-registered with the microCT images. Next, images were analyzed and quantified using AMIDE software (version 1.0.4). The mean tissue activity concentration (μCi/mL) of [¹⁸F] fluoride ions was determined by standardized cylindrical ROIs drawn on distal femurs and lumbar vertebrae, and normalized to the injected dose (μCi). Statistical analysis was performed in the distal femurs and terminal lumbar vertebral body, the most commonly examined bone sites.

2.2.4 Post-Mortem High-Resolution microCT Evaluation

Animals were sacrificed at 4 weeks post-treatment and harvested for left and right femurs, tibias, humeri, and thoracic and lumbar vertebrae. Samples were fixed in 4% paraformaldehyde (PFA) for 48 hours and stored in 70% ethanol for microCT, histological, and immunohistochemical analyses. Femurs were scanned using a high-resolution microCT (SkyScan 1172, Bruker MicroCT N.V., Kontich, Belgium) at an image resolution of 27.4 μm (55 kV and 181 mA radiation source; 0.5-mm aluminum filter). Then, 3D images were reconstructed from the 2D X-ray projections by implementing the Feldkamp algorithm, and appropriate image corrections including ring artifact correction, beam hardening correction, and fine-tuning were processed using NRecon software (SkyScan 1172, Belgium). The dynamic image range (contrast limits) was determined at 0-0.1 in units of attenuation coefficient and applied to all datasets for optimum image contrast.

After acquisition and reconstruction of datasets, images were first reoriented on each 3D plane using DataViewer software (SkyScan 1172, Belgium) to align the long axis of the femur parallel to coronal and sagittal planes. Next, 3D morphometric analyses of the distal femur and the body of lumbar vertebrae were performed using CT-Analyzer software (SkyScan 1172, Belgium). For femurs, the length was divided into ten equal segments between the most proximal point of the growth plate and the proximal end of the third trochanter (1 mm length per segment). The trabecular region was defined as the first three 20 distal segments to include secondary spongiosa in the distal metaphysis. Regions-of-interest (ROIs) were delineated using a freehand drawing tool while maintaining 3.5-pixel clearance from the endosteal surface. A clearance of 0.1 mm was maintained from the growth plate.

A global threshold of 60 (1.01573 g/cm³) was applied to all scans to extract a physiologically accurate representation of the trabecular bone phase. Morphometric parameters were then computed from the binarized images using direct 3D techniques (marching cubes and sphere-fitting methods), and included bone mineral density (BMD, g/cm³), percent bone volume (BV/TV, %), trabecular number (Tb.N, mm⁻¹), trabecular thickness (Tb.Th, mm) and trabecular separation (Tb.Sp, mm). All quantitative and structural parameters followed the nomenclature and units recommended by the American Society for Bone and Mineral Research (ASBMR) Histomorphometry Nomenclature Committee [37]. After data quantification, 3D rendered images were generated to visualize the analyzed regions using the marching cubes method.

### 2.2.5 CFU-F Assay, Histology and Immunohistochemical Analyses

For the colony-forming unit-fibroblast (CFU-F) assay, freshly harvested left and right humeri were flushed to isolate bone marrow stem cells (BMSCs). Isolated marrow cells were seeded on 6-well plates (1×10⁶ cells/well), and cultured for 10 days in Complete MesenCult Medium (STEMCELL Technologies, Inc., Canada) at 37° C. in 5% CO2. CFU-F-derived colonies were stained using Giemsa Staining Solution (EMD Chemicals, Inc., NJ) for 5 minutes and were counted under a microscope.

After microCT scans, the samples were decalcified using 19% EDTA solution for 14 days, dehydrated, and processed for paraffin embedding. Longitudinal sections of 5 (μm thickness were cut on a microtome, and the slides were stained with hematoxylin and eosin (H&E), Trichrome, or with markers of osteoblast (osteocalcin: OCN) and osteoclast (tartrate-resistant acid phosphatase: TRAP) differentiation as previously described [38].

Histological and immunohistochemical specimens were analyzed using an Olympus BX51 microscope (Olympus Corporation, Japan) and photomicrographs were acquired using a MicroFire digital camera with PictureFrame software (Optronics, CA). OCN and TRAP staining were analyzed by three blinded observers and were quantified by the number of osteoblasts per trabecular bone perimeter (N.Ob/B.Pm, mm′¹) and the number of osteoclasts 20 per trabecular bone perimeter (N.Oc/B.Pm, mm⁻¹), respectively. The reported results were the average of data obtained from six random fields per sample.

### 2.3 Statistical Analysis

Means and standard deviations were calculated from numerical data. Statistical analyses were performed using one-way analysis of variance (ANOVA) test for multiple comparisons and Student's t-test for two-group comparisons at 95% confidence levels. Data are presented as mean±SD, with *P<0.05 and **P<0.01.

### 3. Results

### 3.1 Pharmacokinetics Study

### 3.1.1 Serum Pharmacokinetics of NELL-1 and NELL-PEG in Mice

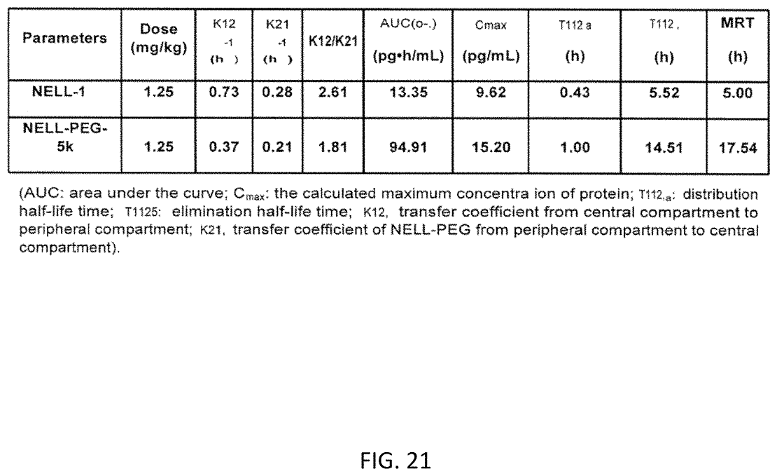

The pharmacokinetic profile of NELL-1 and NELL-PEG were examined in 3-month-old CD-1 mice following an intravenous injection at a dose of 1.25 mg/kg (based on protein content). The protein in serum was quantified using a fluorescence-based protein assay. FIG. 7 shows the concentration change of NELL-PEG and NELL-1 in serum over time after a single intravenous injection in mice. The serum concentration of NELL-1 was significantly increased at various time-points after PEGylation. The pharmacokinetic parameters derived from the serum profiles are shown in FIG. 21. Compared to naked NELL-1, the PEGylated NELL-1 had a significantly higher calculated peak blood concentration (cₘₐₓ). Specifically, the cₘₐₓ of NELL-PEG relative to the naked NELL-1 was about 158%. The protein serum concentration over time can be expressed as:

CNELL-1=8.62e^{-1.60t}+1.00e−0.13t

CNELL-PEG-5k=11.46e^{-0.69t}+3.74e−0.05t

Where t is the time since the injection and _{CNELL-1} ^{and} _{CNELL-PEG-5k} are the protein concentrations following a bolus dose. Accordingly, the total drug exposure (AUC), mean retention time (MRT), distribution half-life time (t½ a), and elimination half-life time (t½ β) of the NELL-PEG appeared longer as well. Compared to naked NELL-1, the relative AUC, MRT, t½ a, and t½ β of NELL-PEG were 710%, 263%, 233%, and 351%, respectively. In addition, the two transfer coefficients, _{K12} ^{and} _{K21}, became smaller, indicating a slower transfer rate between central and peripheral compartments after PEGylation. The transfer coefficient of NELL-PEG from the central compartment to the peripheral compartment _{(K12)} was only 51.4% of that for naked NELL-1, and the reverse transfer coefficient _{(K21)} was 74.1% compared to that of naked NELL-1. Therefore, NELL-PEG presents a more potent NELL-1 type for in vivo pharmacological studies. FIG. 21.

### 3.1.2 Biodistribution of NELL-1 and NELL-PEG in Mice

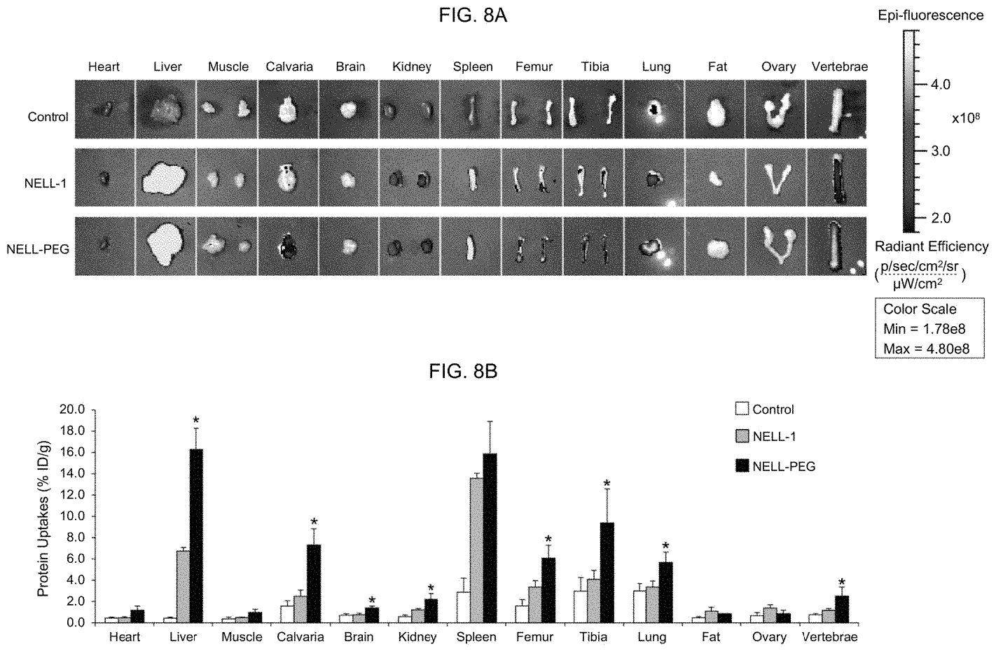

Ex vivo biodistribution of NELL-1 and NELL-PEG were examined in CD-1 mice at 48 h. The protein was labeled with VivoTag 680XL first, and then administered to the mice at a 5 dose of 1.25 mg/kg (based on protein content). The organs were collected at 48 h and imaged by the IVIS imaging system. The results are shown in FIGS. 8A-B. The NELL-PEG group had significantly higher uptakes in some but not all of the tissues examined. The protein uptake in the heart, muscle, brain, spleen, fat, and ovary were similar for NELL-PEG and NELL-1, and the accumulation in these organs except the spleen was negligible compared to the other organs. At 48 h after IV injection, the spleen had the greatest uptake of NELL-1 whereas the liver had the greatest uptake of NELL-PEG. The uptake amount in the liver varied from 6.7±0.33 to 16.3±1.98% ID/g before and after PEGylation, showing a significant difference. For the spleen tissue, the corresponding uptake values were 13.6±0.47 and 15.9±3.04% ID/g, displaying no statistical difference. However, protein uptakes in the bones (calvaria, femur, tibia, vertebrae) were significantly increased after PEGylation. The amount of NELL-PEG in calvaria, femur, tibia, and vertebrae relative to NELL-1 were 294%, 181%, 229%, and 215%, respectively, showing greater NELL-PEG protein distribution to the bone target tissues.

### 3.2 Systemic Osteogenic Potential Study (at q4d and q7d Injection Schedules)

### 3.2.1 In Vivo Bone Densitometry

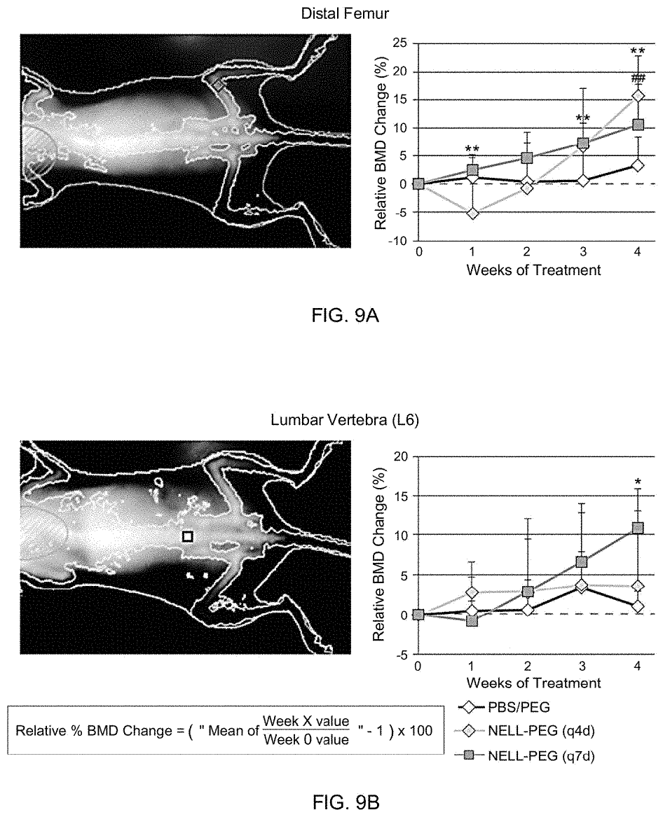

The BMD change in mice femurs and lumbar vertebrae were monitored weekly by DXA. Results were expressed as percent changes in areal BMD relative to the respective pretreatment values at week 0. For the PBS/PEG control groups, BMD remained steady at baseline levels in distal femurs and lumbar vertebrae (L6) for the experimental period. In contrast, the NELL-PEG groups (both q4d and q7d) displayed a gradual and significant increase in BMD (16% and 11% respectively) of the distal femurs by 4 week post-treatment compared to the pre-treatment values but with no significant difference between the q4d and q7d groups (FIG. 9A). The lumbar vertebrae in both NELL-PEG groups exhibited increasing BMD relative to the baseline throughout the experiment. Interestingly, the q7d NELL-PEG group showed a greater BMD increment (11%) than the q4d NELL-PEG group (4%) at week 4. No significant difference in lumbar BMD was observed between the two experimental groups at week 4 (FIG. 9B).

3.2.2 In Vivo microPET/CT Bone Scan Using [¹⁸F] Fluoride Ion

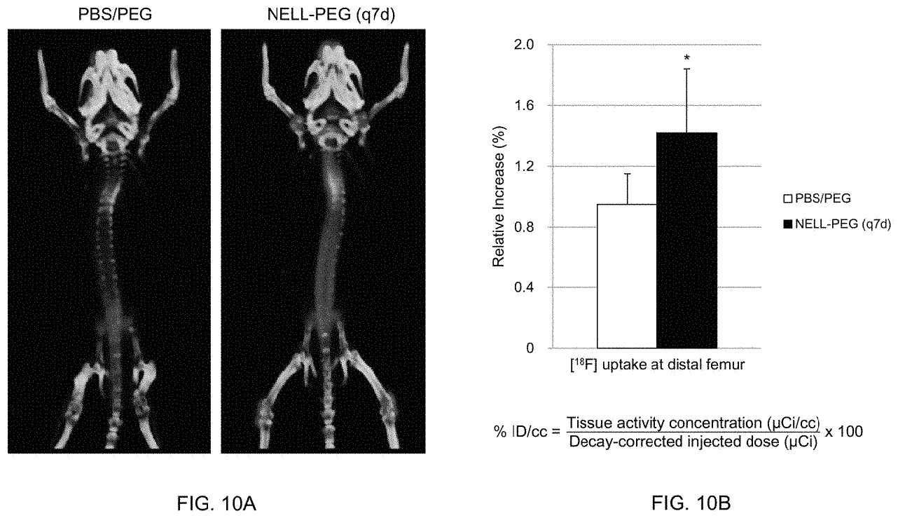

The physiological bone metabolic activity of the mouse skeleton was examined by weekly microPET/CT combination scans using [¹⁸F] fluoride ion. Qualitative analysis of live-microCT images revealed increased BMD in the NELL-PEG (q7d) group at the overall skeletal sites compared to control mice (data not shown). Live-[¹⁸F] microPET quantification data at each time-point were expressed as the percent of decay-corrected injected activity per cc of tissue (% ID/cc), using the formula shown in FIG. 10A, from which normalized mean values were then generated. MicroPET/CT revealed increased new bone formation in the NELL-PEG (q7d) group with higher activity distribution particularly near growth plate areas in the vertebral column, proximal humeri, proximal and distal femurs, and proximal tibias (FIG. 10A). The distal femur showed a significantly increased new bone formation after 4 weeks of treatment (FIG. 10B), and the terminal lumbar vertebra (L6) showed a mean increase in the NELL-PEG (q7d) group compared to control (data not shown).

3.2.3 Post-Mortem High-Resolution microCT Evaluation

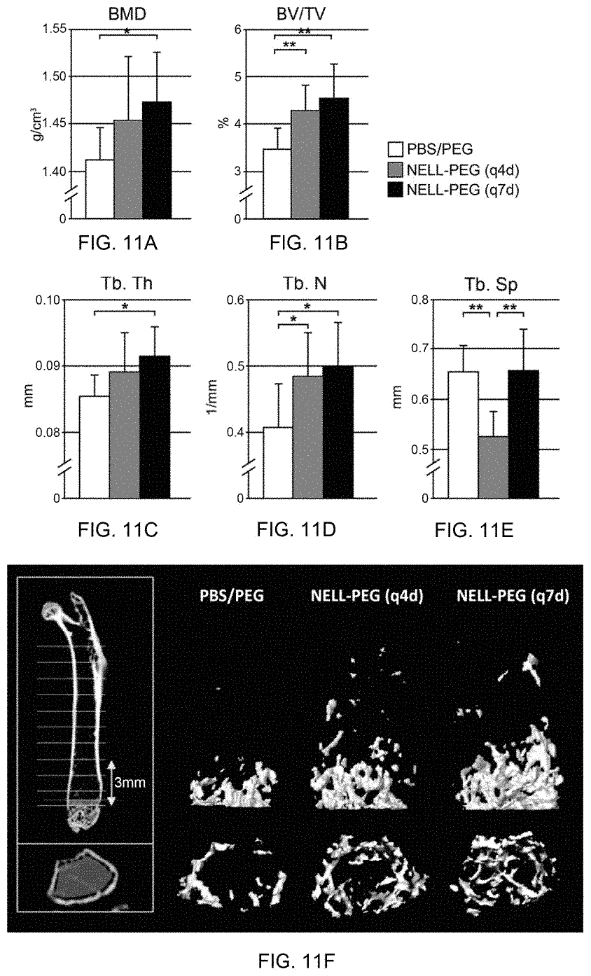

In line with the in vivo DXA and microPET bone scan results, post-mortem microCT confirmed considerable improvements in the trabecular bone density (BMD), bone volume fraction (BV/TV), and structural parameters (Tb.Th, Tb.N, Tb.Sp) in femurs of both q4d and q7d NELL-PEG groups at 4 weeks post-treatment compared to the corresponding PBS/PEG control (FIGS. 11A-E). Both NELL-PEG groups demonstrated a statistically significant increase in bone volume fraction (BV/TV) compared to the control, but with no substantial difference between each other (FIG. 1 IB). Notably, the q7d NELL-PEG group also showed significant improvement in other trabecular parameters (BMD, Tb.Th, and Tb.N) compared to the control (FIGS. 11A-E).

### 3.2.4 CFU-F Assay, Histological and Immunohistochemical Analyses

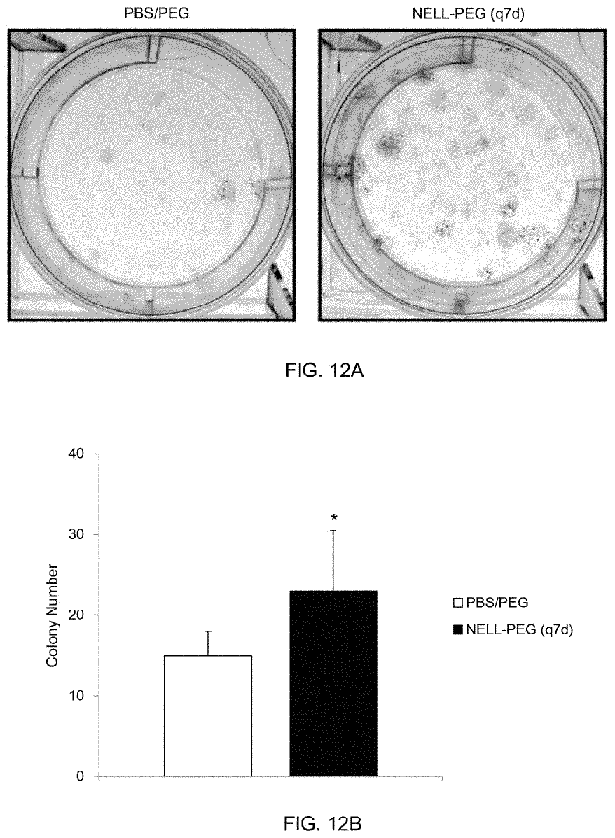

To examine the effect of NELL-1 on bone marrow stromal cell (BMSC) content, a CFUF assay was performed using fresh and passaged bone marrow isolated from humeri immediately post-harvest. The results at 4 weeks post-treatment displayed a statistically significant increase in BMSC numbers in the NELL-PEG (q7d) group compared to the control, suggesting that NELL-PEG enhances the proliferation of BMSCs when administered systemically (FIG. 12).

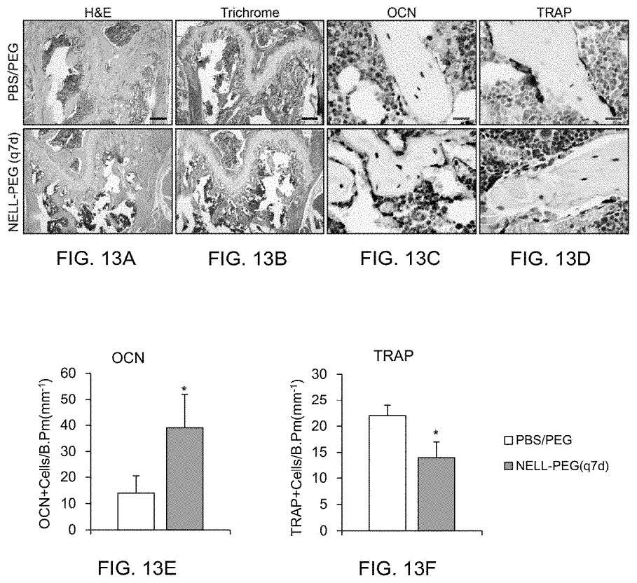

Histology stain confirmed increased bone formation and trabeculation in the metaphyseal area of the distal femur in the NELL-PEG treatment group (FIGS. 13A, B). Consistently, OCN immunostaining demonstrated increased osteoblast numbers while TRAP staining demonstrated decreased osteoclast numbers in the NELL-PEG-treated group compared to the PBS/PEG control group (FIGS. 13C-F).

### 4. Discussion

Osteoporosis, characterized by decreased bone mass and a deterioration of bone microarchitecture due to increased osteoclastic and decreased osteoblastic activities, is a common metabolic bone disease with associated bone fragility and increased risk of fracture. Therapeutic approaches to osteoporotic bone loss have focused on either anabolic or antiresorptive agents, with many new biologies that increase Wnt/β-catenin activity currently in development. NELL-1, one such biologic, has proven successful as a growth factor-based local therapeutic similar to BMP-2, the most commonly used osteogenic growth factor in the market. It is important to note, however, that NELL-1 demonstrated fewer side effects when administered in vivo in comparison to BMP-2 [38, 39] as a direct result of its specificity to osteochondral lineage cells. With a previous genome-wide linkage study describing an association between NELL-1 and osteoporosis [28], with NELL-1's dual anabolic and antiresorptive properties described both in vivo and in vitro [19], and with the recent success of reversing OVX-induced bone loss in mice via IV administration of naked NELL-1 [30], we have been both motivated and challenged to further investigate NELL-1 as a systemic antiosteoporosis therapeutic. Thus, the present study examined the implementation of PEGylation—the most established, FDA-approved technique to prolong a protein's half-life—to develop NELL-1 as an effective systemic therapy.

In our previous study, three types of PEG (linear 5 KDa, linear 20 kDa and branched 40 KDa) were investigated for their effects on the stability and bioactivity of NELL-1 in vitro. NELLPEG-5k was selected for the current study because it demonstrated the highest thermostability among the three types of NELL-PEG tested and a bioactivity comparable to that of naked NELL-1 [35]. In this study, we investigated whether the PEGylation would improve the pharmacokinetics and bone quality of NELL-1 in vivo when intravenously administered. To achieve this, the study was performed with two aims. The first aim was to evaluate the serum protein concentration with time and biodistribution of NELL-PEG in comparison to naked NELL-1. The second aim was to investigate the systemic osteogenic capacity of NELL-PEG in vivo when administered at two different injection schedules, q4d and q7d, after performing an in vivo pharmacokinetic assessment.

The pharmacokinetics of PEGylated NELL-1 was investigated first. After being injected intravenously, NELL-PEG rapidly distributed throughout the vasculature and then began to clear from the blood system. The serum concentration and time course of NELL-PEG is shown in FIG. 7. Meanwhile, NELL-PEG slowly extravasated and accumulated in specific organ regions, and we assessed the biodistribution of NELL-PEG by an ex vivo method at 48 h as illustrated in FIGS. 8A-B. The two parts consist of the pharmacokinetic process of NELL-PEG in vivo.

The NELL-PEG group had higher exposure AUC, greater maximum concentration Cmax, and longer half-life time _{(T1/2)} compared to the corresponding values for the naked NELL-1, which can be attributed to the decreased renal clearance after PEGylation. This result is in agreement with previous studies of PEGylated proteins [40-42]. Furthermore, the smaller _{K12/K21} ratio of NELL-PEG indicated that its transfer from the central compartment to the peripheral compartment was reduced by conjugation with PEG. The transfer rate became slower for both directions; in particular, the transfer rate of NELL-PEG was only half that of the naked NELL-1 for the transfer from vascular to extravascular compartments, indicating a high retention in the central compartment. This was confirmed by the high mean retention time (MRT), maximum serum concentration _{(Cmax)}, and AUC of NELL-PEG. The changes in the pharmacokinetic parameters after PEGylation could lead to increased clinical effectiveness of NELL-1 since it can maintain a protein concentration above the minimum effective concentration for an extended period of time.

PEGylation may also influence the biodistribution of protein after extravasation [43]. NELLPEG and NELL-1 protein were fluorescently labeled and administered in mice, and the organs examined via imaging after 48 hours. This result indicated that NELL-PEG had significantly higher uptake in bone tissue compared to the naked NELL-1 after a single IV dose. This finding may be attributable to two reasons. First, the PEGylated NELL-1 has a higher concentration and longer retention time in the vasculature system as confirmed in the pharmacokinetics study, thus forming a higher concentration gradient between the blood vessel and tissue which in turn lead to a higher diffusion rate and eventually a higher uptake of protein. Second, NELL-1 could bind specifically to the receptor of apoptosis related protein 3 (APR3) membrane protein on osteoblasts in bone tissue [44], thereby resulting in a greater accumulation compared to other peripheral tissues such as muscle and adipose tissue. Although the highest concentration of protein was found in the liver and spleen 48 h after IV injection, autopsy and histology findings revealed no pathology at high NELL-PEG concentrations up to 50 ug/mL (data not shown). More importantly, NELL-PEG distribution in vital organs such as the brain and heart remained at low levels and did not exhibit a significant increase after PEGylation. Therefore, the pharmacokinetics study indicated that the PEGylation significantly affected the clearance process and tissue penetration of NELL-1. Such a favorable accumulation of the protein in bone tissue sheds positive light on the benefits of NELL-1 as a systemic therapy for skeletal diseases.

Since the PEGylation of NELL-1 significantly improved its pharmacokinetics, we further investigated the systemic osteogenic capacity of NELL-PEG in vivo. The systemic administration of NELL-PEG in mice for 4 weeks revealed significantly increased bone mineral density (based on DXA) and percent bone volume (based on microCT) in 3-month-old mice in both q4d and q7d injection schedules. While the q4d and q7d NELL-PEG groups did not exhibit notable differences between each other, both groups exhibited significant improvements in all measured DXA and microCT parameters compared to the PBS/PEG control including trabecular bone density (BMD), volume (BV/TV), and structural measurements (Tb.Th, Tb.N, Tb.Sp). Interestingly, we found that although not statistically significant, the q7d group exhibited a greater osteogenic effect compared to the q4d group. Speculated reasons for these findings that invite further investigation include that this strain of mouse is reported to exhibit a poor response to treatment or OVX in the vertebral body due to very low baseline bone levels [45,46]. In addition, the overdosing or frequent injection of a growth factor-based drug such as PTH has been shown to down-regulate the osteogenic effect, possibly by initiating negative feedback mechanism by overloading the receptor [47]. This is evidenced by the fact that pulsatile PTH dosing yields a favorable outcome in improving bone growth whereas continuous PTH dosing results in net bone loss [48,49].

Moreover, F-18 microPET scanning of the NELL-PEG (q7d) group 4 weeks post-treatment revealed an increased new bone formation particularly in the vertebral column, proximal humeri, proximal and distal femurs, and proximal tibias compared to the control. Furthermore, a colony-forming unit (CFU) assay examining the mesenchymal cell (MSC) content in the humeri demonstrated a statistically significant increase in MSC numbers in the NELL-PEG (q7d) group compared to the control, suggesting that NELL-PEG enhances the proliferation of BMSCs when administered systemically q7d. Histology and immunohistochemistry analyses further corroborated increased bone formation and trabeculation as well as increased osteoblastic and decreased osteoclastic activities in the NELL-PEG group.

Taken together, these exciting findings encourage us to further investigate NELL-PEG as a systemic anti-osteoporosis therapeutic with not just a clinically feasible, but in fact a substantially improved injection schedule that could reduce costs and improve patient compliance in chronic treatment clinical settings. With regards to the circulation time of NELL-1 in vivo, an injection frequency reduced to q4d would make NELL-PEG a systemic therapy comparable to that of other combined anabolic and antiresorptive agents currently in development, specifically anti-DKK1 or anti-Sclerostin [36]. An injection frequency further reduced to q7d as suggested in this present study, however, would present a superior injection schedule when compared to other systemic anti-osteoporosis therapeutics. Such a reduced injection schedule may lead to significantly reduced treatment costs and increased patient compliance in clinical settings for the treatment of osteoporosis and other osteodeficient disorders. Furthermore, the PEGylation of osteoinductive molecule NELL-1 as a systemic therapy may also aid in the treatment of large post-surgical orthopedic and craniofacial defects such as in cleft palate and cranial suture repair.

### 5. Conclusions

The presented success in the PEGylation of NELL-1 is both innovative and novel in the field of orthopedic research. With an improved biodistribution and pharmacokinetics in vivo and with enhanced bone quality after systemic administration, this exciting discovery of a reduced injection frequency of NELL-PEG to q7d demonstrates PEGylated NELL-1 to be a strong candidate as a translational, systemic therapeutic for various osteodeficient disorders such as osteoporosis and large post-surgical orthopedic and craniofacial defects. Moreover, the present study champions the PEGylation method to be a revolutionary platform technology that invites further investigation of novel methodologies to deliver additional osteogenic growth factors.

### REFERENCES

[1] Heinemann D F. Osteoporosis. An overview of the National Osteoporosis Foundation clinical practice guide. Geriatrics. 2000; 55:31-6; quiz 9.
[2] Berry S D, Kiel D P, Donaldson M G, Cummings S R, Kanis J A, Johansson H, et al. Application of the National Osteoporosis Foundation Guidelines to postmenopausal women and men: the Framingham Osteoporosis Study. Osteoporos Int. 2010; 21:53-60.
[3] Shuler F D, Conjeski J, Kendall D, Salava J. Understanding the burden of osteoporosis and use of the World Health Organization FRAX. Orthopedics. 2012; 35:798-805.
[4] Watts N B, Lewiecki E M, Miller P D, Balm S. National Osteoporosis Foundation 2008 Clinician's Guide to Prevention and Treatment of Osteoporosis and the World Health Organization Fracture Risk Assessment Tool (FRAX): what they mean to the bone densitometrist and bone technologist. J Clin Densitom. 2008; 11:473-7.
[5] Czerwinski E, Badurski J E, Marcinowska-Suchowierska E, Osieleniec J. Current understanding of osteoporosis according to the position of the World Health Organization (WHO) and International Osteoporosis Foundation. Ortop Traumatol Rehabil. 2007; 9:337-56.
[6] Jilka R L. Biology of the basic multicellular unit and the pathophysiology of osteoporosis. Med Pediatr Oncol. 2003; 41:182-5.
[7] Cusano N E, Bilezikian J P. Combination anabolic and antiresorptive therapy for osteoporosis. Endocrinol Metab Clin North Am. 2012; 41:643-54.
[8] Bilezikian J P. Combination anabolic and antiresorptive therapy for osteoporosis: opening the anabolic window. Curr Osteoporos Rep. 2008; 6:24-30.
[9] Vahle J L, Sato M, Long G G, Young J K, Francis P C, Engelhardt J A, et al. Skeletal changes in rats given daily subcutaneous injections of recombinant human parathyroid hormone (1-34) for 2 years and relevance to human safety. Toxicol Pathol. 2002; 30:312-21.
[10] Jilka R L. Molecular and cellular mechanisms of the anabolic effect of intermittent PTH.

Bone. 2007; 40:1434-46.
[11] Neer R M, Amaud C D, Zanchetta J R, Prince R, Gaich G A, Reginster J Y, et al. Effect of parathyroid hormone (1-34) on fractures and bone mineral density in postmenopausal women with osteoporosis. N Engl J Med. 2001; 344:1434-41.
[12] Minear S, Leucht P, Jiang J, Liu B, Zeng A, Fuerer C, et al. Wnt proteins promote bone regeneration. Sci Transl Med. 2010; 2:29ra30.
[13] Wagner E R, Zhu G, Zhang B Q, Luo Q, Shi Q, Huang E, et al. The therapeutic potential of the Wnt signaling pathway in bone disorders. Curr Mol Pharmacol. 2011; 4:14-25.
[14] Cabrera C V, Alonso M C, Johnston P, Phillips R G, Lawrence P A. Phenocopies induced with antisense RNA identify the wingless gene. Cell. 1987; 50:659-63.
[15] Nusslein-Volhard C, Wieschaus E. Mutations affecting segment number and polarity in Drosophila. Nature. 1980; 287:795-801.
[16] Ting K, Vastardis H, Mulliken J B, Soo C, Tieu A, Do H, et al. Human NELL-1 expressed in unilateral coronal synostosis. Journal of bone and mineral research: the official journal of the American Society for Bone and Mineral Research. 1999; 14:80-9.
[17] Aghaloo T, Cowan C M, Chou Y F, Zhang X, Lee H, Miao S, et al. Nell-1-induced bone regeneration in calvarial defects. Am J Pathol. 2006; 169:903-15.
[18] Cowan C M, Zhang X, James A W, Kim T M, Sun N, Wu B, et al. NELL-1 increases pre-osteoblast mineralization using both phosphate transporter Pit1 and Pit2. Biochem Biophys Res Commun. 2012; 422:351-7.
[19] James A W S J, Velasco O, Asatrian G, Chung G, Khadarian K, Zhang Y, Chang L, Goyal R, Kim J, Zhang X, Adams J, Ting K, Soo C. Systemic administration of NELL-1, a Wnt/β-Catenin Regulator, Induces Bone Formation in Osteoporotic Mice via Integrinβ1. American Society of Bone and Mineral Research (ASBMR) Annual Meeting 2013.
[20] Kwak J, Zara J N, Chiang M, Ngo R, Shen J, James A W, et al. NELL-1 injection maintains long-bone quantity and quality in an ovariectomy-induced osteoporotic senile rat model. Tissue Eng Part A. 2013; 19:426-36.
[21] Li W, Lee M, Whang J, Siu R K, Zhang X, Liu C, et al. Delivery of lyophilized Nell-1 in a rat spinal fusion model. Tissue Eng Part A. 2010; 16:2861-70.
[22] Lu S S, Zhang X, Soo C, Hsu T, Napoli A, Aghaloo T, et al. The osteoinductive properties of Nell-1 in a rat spinal fusion model. The spine journal: official journal of the North American Spine Society. 2007; 7:50-60.
[23] Siu R K, Lu S S, Li W, Whang J, McNeill G, Zhang X, et al. Nell-1 protein promotes bone formation in a sheep spinal fusion model. Tissue Eng Part A. 2011; 17:1123-35.
[24] Aghaloo T, Jiang X, Soo C, Zhang Z, Zhang X, Hu J, et al. A study of the role of nell-1 gene modified goat bone marrow stromal cells in promoting new bone formation. Mol Ther. 2007; 15:1872-80.
[25] Aghaloo T, Cowan C M, Zhang X, Freymiller E, Soo C, Wu B, et al. The effect 20 of NELLI and bone morphogenetic protein-2 on calvarial bone regeneration. J Oral Maxillofac Surg. 2010; 68:300-8.
[26] Zhang X, Ting K, Bessette C M, Culiat C T, Sung S J, Lee H, et al. Nell-1, a key functional mediator of Runx2, partially rescues calvarial defects in Runx2(+/−) mice. Journal of bone and mineral research: the official journal of the American Society for Bone and Mineral Research. 2011; 26:777-91.
[27] James A W, Pan A, Chiang M, Zara J, Zhang X, Ting K, et al. A new function of Nell-1 protein in repressing adipogenic differentiation. Biochem Biophys Res Commun. 2011.
[28] Karasik D, Hsu Y H, Zhou Y, Cupples L A, Kiel D P, Demissie S. Genome-wide pleiotropy of osteoporosis-related phenotypes: the Framingham Study. Journal of bone and mineral research: the official journal of the American Society for Bone and Mineral Research. 2010; 25:1555-63.
[29] James A W S J, Zhang X, Asatrian G, Goyal R, Bengs B, Culiat C T, Turner A S, Seim H B, Lyons K, Adams J S, Ting K, Soo C. NELL-1 in the Etiopathogenesis and Treatment of Osteoporotic Bone Loss. Manuscript in revision.
[30] Asatrian G, James A W, Chung C, Velasco O, Zhang X, Ting K, et al. Systemic administration of the osteoinductive growth factor NELL-1 promotes bone formation in osteoporotic Mice. Military Health System Research Symposium (Conference). 2013.
[31] Jevsevar S, Kunstelj M, Porekar V G. PEGylation of therapeutic proteins. Biotechnol J. 2010; 5:113-28.
[32] Ng E W, Shima D T, Calias P, Cunningham E T, Jr., Guyer D R, Adamis A P. Pegaptanib, a targeted antiVEGF aptamer for ocular vascular disease. Nat Rev Drug Discov. 2006; 5:123-32.
[33] Levy Y, Hershfield M S, Femandez-Mejia C, Polmar S H, Scudiery D, Berger M, et al. Adenosine deaminase deficiency with late onset of recurrent infections: response to treatment with polyethylene glycol-modified adenosine deaminase. J Pediatr. 1988; 113:312-7.
[34] Pfister D, Morbidelli M. Process for protein PEGylation. Journal of controlled release: official journal of the Controlled Release Society. 2014; 180:134-49.
[35] Zhang Y, Velasco O, Zhang X, Ting K, Soo C, Wu B M. Bioactivity and circulation time of PEGylated NELL-1 in mice and the potential for osteoporosis therapy. Biomaterials. 2014; 35:6614-21.
[36] Glantschnig H, Scott K, Hampton R, Wei N, McCracken P, Nantermet P, et al. A rate-limiting role for Dickkopf-1 in bone formation and the remediation of bone loss in mouse and primate models of postmenopausal osteoporosis by an experimental therapeutic antibody. J Pharmacol Exp Ther. 2011; 338:568-78.
[37] Parfitt A M, Drezner M K, Glorieux F H, Kanis J A, Malluche H, Meunier P J, et al. Bone histomorphometry; standardization of nomenclature, symbols, and units. Report of the ASBMR Histomorphometry Nomenclature Committee. Journal of bone and mineral research: the official journal of the American Society for Bone and Mineral Research. 1987; 2:595-610.
[38] Shen J, James A W, Zara J N, Asatrian G, Khadarian K, Zhang J B, et al. BMP2-induced inflammation can be suppressed by the osteoinductive growth factor NELL-1. Tissue engineering Part A. 2013; 19:2390-401.
[39] Zhang X, Zara J, Siu R K, Ting K, Soo C. The role of NELL-1, a growth factor associated with craniosynostosis, in promoting bone regeneration. Journal of dental research. 2010; 89:865-78.
[40] Bansal R, Post E, Proost J H, de Jager-Krikken A, Poelstra K, Pralcash J. PEGylation improves pharmacokinetic profile, liver uptake and efficacy of Interferon gamma in liver fibrosis. Journal of controlled release: official journal of the Controlled Release Society. 2011; 154:233-40.
[41] Mattos A, de Jager-Krikken A, de Haan M, Beljaars L, Poelstra K. PEGylation of interleukin-10 improves the pharmacokinetic profile and enhances the antifibrotic effectivity in CCl(4)-induced fibrogenesis in mice. Journal of controlled release: official journal of the Controlled Release Society. 2012; 162:84-91.
[42] Zhang C, Fan K, Luo H, Ma X, Liu R, Yang L, et al. Characterization, efficacy, pharmacokinetics, and biodistribution of 5 kDa mPEG modified tetrameric canine uricase variant. International journal of pharmaceutics. 2012; 430:307-17.
[43] Caliceti P, Schiavon O, Veronese FM. Biopharmaceutical properties of uricase conjugated to neutral and amphiphilic polymers. Bioconjugate chemistry. 1999; 10:638-46.
[44] Zou X, Shen J, Chen F, Ting K, Zheng Z, Pang S, et al. NELL-1 binds to APR3 affecting human osteoblast proliferation and differentiation. FEBS letters. 2011; 585:2410-8.
[45] Li C Y, Schaffler M B, Wolde-Semait H T, Hernandez C J, Jepsen K J. Genetic background influences cortical bone response to ovariectomy. Journal of bone and mineral research: the official journal of the American Society for Bone and Mineral Research. 2005; 20:2150-8.
[46] Bouxsein M L, Myers K S, Shultz K L, Donahue L R, Rosen C J, Beamer W G. Ovariectomy-induced bone loss varies among inbred strains of mice. Journal of bone and mineral research: the official journal of the American Society for Bone and Mineral Research. 2005; 20:1085-92.
[47] Zhou H, Iida-Klein A, Lu S S, Ducayen-Knowles M, Levine L R, Dempster D W, et al. Anabolic action of parathyroid hormone on cortical and cancellous bone differs between axial and appendicular skeletal sites in mice. Bone. 2003; 32:513-20.
[48] Potter L K, Greller L D, Cho C R, Nuttall M E, Stroup G B, Suva L J, et al. Response to continuous and pulsatile PTH dosing: a mathematical model for parathyroid hormone receptor kinetics. Bone. 2005; 37:159-69.
[49] Schmitt C P, Hessing S, Oh J, Weber L, Ochlich P, Mehls O. Intermittent administration of parathyroid hormone (1-37) improves growth and bone mineral density in uremic rats. Kidney international. 2000; 57:1484-92.

### Example 3. Systemic Delivery of Chemically Modified NELL-1 Promotes Bone Formation in Osteoporotic Mice

### 1. Introduction

Nel-like molecule-1 (NELL-1) is a pro-ostegenic, secretory molecule first identified in patients with craniosynostosis. NELL-1 exhibits significant bone forming effects when locally implanted in calvarial, axial and appendicular defects in both small and large animal models. Here, we investigate the systemic application of NELL-1 (intravenous delivery) on reversing osteoporosis in ovariectomized (OVX) mice. Furthermore, we evaluate the effect of PEGylation, an established chemical process of attaching polyethylene glycol (PEG) to improve molecular physiochemistry, on NELL-1's half-life and bioactivity.

### 2. Methods

First, we evaluated the efficacy of intravenous NELL-1 administration in an OVX-induced osteoporotic mouse model. After induction of osteoporosis, both OVX and Sham-treated animals were administered either PBS or NELL-1 (1.25 ug/mg), q48 hours, via tail injection. Detailed analyses included dual energy X-ray (DEXA), liveCT/PET, microCT, histology, and immunohistochemistry. Next, we examine the effects of various PEGylation techniques (5K linear, 20K linear, and 40K branched) on NELL-1 and their effects on systemic half-life and in vitro bioactivity in the Saos2 cell line.

### 3. Results

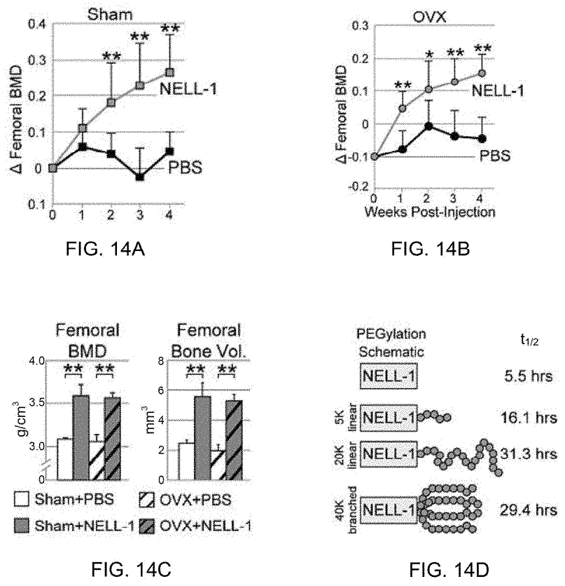

OVX induction of osteoporosis was confirmed by an 11% mean reduction in BMD, at which time intravenous NELL-1 therapy was instituted. After 4 weeks of systemic administration, NELL-1 treatment resulted in a significant increase in BMD in both OVX and Sham-operated animals (FIGS. 14A,B). Post-mortem microCT examination of distal femurs and lumbar spines confirmed that animals treated with NELL-1 possessed increased bone density and bone volume (FIG. 14C). Furthermore, immunohistochemistry of NELL-1-treated femurs exhibited increased Osteocalcin (OCN) and reduced TRAP (tartrate-resistant acid phosphatase) staining, indicative of increased bone formation and inhibition of osteoclast activity. Next, we evaluated the effects of PEGylation on NELL-1 half-life and bioactivity. Linear PEGylation with 5K Da and 20K Da increased half-life of NELL-1 from 5.5 hours to 16.1 and 31.3 hours, respectively. When attaching 40K Da of branched PEG to NELL-1, half-life was 29.4 hours (FIG. 14D). Importantly, Alizarin red staining for mineralization confirmed that all forms of PEGylation did not significantly reduce in vitro osteogenic induction.

### 4. Conclusion

NELL-1 is a pro-osteogenic, anti-resorptive cytokine, which can promote bone formation upon intravenous administration. Thus, systemic administration of NELL-1 holds promise for future clinical application in the treatment of osteoporotic bone loss. Additionally, we observed that NELL-1's pharmacokinetic parameters can be improved by PEGylation, without a negative impact on its osteoinductive activity.

### Example 4. Chemical Modification of NELL-1 Protein for Systemic Treatment of Osteoporosis in Mice

### 1. Introduction

Nel-like protein 1 (NELL-1) is a pro-osteogenic secreted cytokine first discovered by its overexpression in craniosynostosis patients. Subsequently, NELL-1 has been studied for its local bone forming potential, and has been shown to promote endochondral and intramembranous ossification, leading to re-ossification of critical sized defects in small and large animal models. For example, in rats, NELL-1 has been shown to heal critical-sized femoral defects as well as induce posterolateral spinal fusion. These findings have been translated to large animals, having shown that NELL-1 can induce spinal fusion between vertebrae in sheep and non-human primates. Mechanistically, NELL-1 is known to regulate Runt-related transcription factor-2 (Runx2) activity and phosphorylation. This is achieved by activation of Wnt/Beta-Catenin signaling via binding to IntegrinB 1. Further, NELL-1 has been shown to not only activate osteoblasts, but also to suppress osteoclast activity. This dual anabolic, anti-osteoclastic effect sparked interest in the use of NELL-1 treatment for osteoporosis. To this end, NELL-1 was administered via intravenous tail injection in ovariectomy (OVX)-induced osteoporotic mice. Furthermore, we investigate the effects of PEGylation (a chemical process of attaching polyethylene glycol (PEG) to improve molecular physiochemistry) on NELL-1's systemic half-life.

### 2. Method

To assess the systemic effect of NELL-1 in both osteoporotic and non-osteoporotic mice, B6 mice (N=28) were randomly distributed into four treatment groups: 1] Sham 20 surgery+PBS(phosphate buffered saline); 2] Sham surgery+NELL-1; 3] OVX+PBS; 4] OVX+NELL-1. To monitor bone mineral density (BMD), dual-energy X-Ray absorptiometry (DXA) was performed weekly throughout the study period, with regions of interest (ROI) being the distal femur and lumbar spine. After a five-week induction period post OVX, systemic NELL-1 therapy via intravenous tail vein injection was initiated, with either PBS or 25 NELL-1 (1.25 ug/kg q48 hours). LiveCT and PET (positron emission tomography) scans were performed biweekly during systemic treatment. Animals were sacrificed at four weeks post systemic therapy. Analyses included high-resolution micro Computed Tomography (microCT), histology and immunohistochemistry with for markers of osteoblasts (Osteocalcin (OCN), Osteopontin (OPN)) and osteoclasts (Tartrate-resistant acid phosphatase (TRAP)). Next, the effects of PEGylation on NELL-1's osteogenic potential were investigated using various PEGylation techniques, including 5K linear, 20K linear, and 40K branched. The systemic half-life of chemically modified NELL-1 was assessed using FITC conjugated NELL-1. Osteogenic activity was assessed by in vitro mineralization studies in the osteoblast cell line, Saos2. Statistical analyses were performed using one-way ANOVA, followed by post-hoc Tukey's tests to compare two groups.

### 3. Results

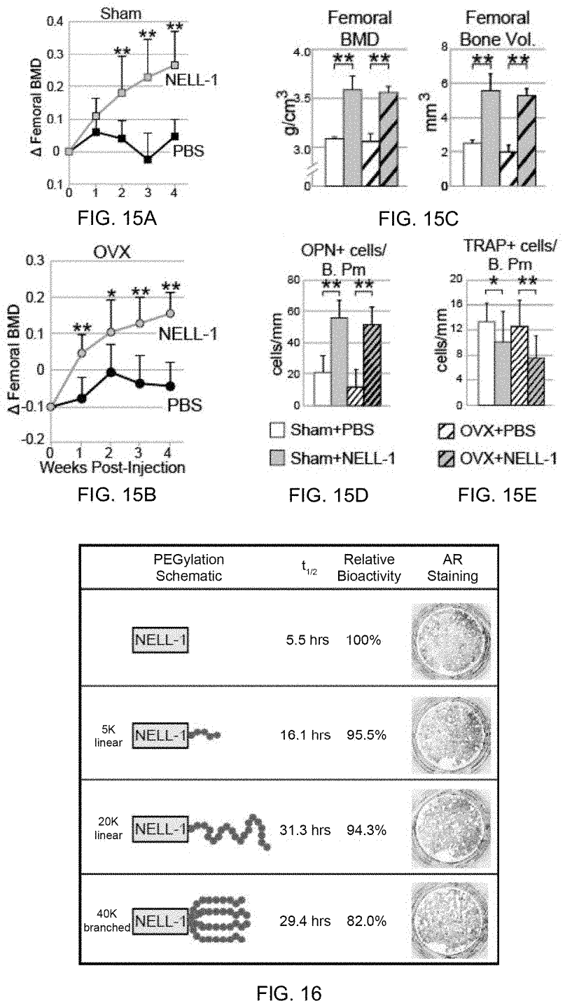

Osteoporosis induction was confirmed by an 11% mean reduction in BMD, upon which intravenous NELL-1 therapy was instituted. IV NELL-1 treatment resulted in a gradual increase in BMD in both Sham- and OVX-treated animals (FIGS. 15A, B). After four weeks NELL-1 treatment, a 26% and 29% increase in mean BMD was observed in the femur and lumbar spine, respectively. MicroCT analysis confirmed a significant increase in all parameters of interest, including BMD, bone volume, and trabecular thickness, observed in both non-osteoporotic and osteoporotic animals (FIG. 15C). Further, histomorphometric studies of H&E-stained samples revealed significantly higher bone area in NELL-1-treated samples. Increased osteoblasts per bone perimeter (B. Pm) were observed with NELL-1 treatment, as confirmed by increased immunostaining for OCN and OPN (FIG. 15D). Conversely, NELL-1 treatment inhibited osteoclast numbers, as observed by decreased TRAP staining (FIG. 15E). Next, we evaluated the effects of PEGylation on NELL-1 half-life and bioactivity (FIG. 16). PEGylation with Linear 5K Da, Linear 20K, and Branched 40K was observed to increase the half-life of NELL-1 from 5.5 hours to 16.1, 31.3, and 29.4 hours, respectively. Importantly, Alizarin red staining for mineralization confirmed that all forms of PEGylation significantly induced in vitro osteogenesis.

### 4. Discussion

NELL-1 is a pro-osteogenic cytokine capable of not only inducing osteoblastogenesis but able to inhibit osteoclastic activity. Here, we have shown that systemic treatment of NELL-1 is efficacious in the reversal of osteoporotic bone loss in mice. Further, we observed that PEGylation is capable of increasing NELL-1's half-life by three to five-fold without detrimental effects on its osteogenic potential. Ongoing studies are being performed to investigate the effect of PEGylated NELL-1 in vivo. In aggregate, these findings suggest that NELL-1 based therapies may hold clinical promise for future application in the prevention and reversal of osteoporotic bone loss.

### Example 5. Intravenously Administered PEG-NELL-1 Promotes Bone Formation and Density

With an aging population, the biomedical burden of osteoporosis is significantly escalating, with no novel therapeutic to address systemic bone loss. NELL-1 is an osteoinductive factor recently discovered to induce bone formation and reverse osteoporotic bone loss when administered intravenously. However, unmodified NELL-1 requires an impractical 48 hr injection frequency and thus limits NELL-1's translation into a clinical setting. Here, we investigate the potential of PEG (poly-ethylene glycol) addition to NELL-1 to improve pharmacokinetics while maintaining osteogenic potential.

First, we assayed 3 PEGylation patterns (5K-linear, 20K-linear, and 40K-branched) to investigate the systemic bioavailability and in vitro osteogenic potential. We next sought to investigate the potential of systemically administered PEG-NELL-1 (q4d) to promote bone formation in vivo in comparison to control (3mo, CD-1 mice; n=10). Animals' bone mineral density (BMD) was monitored weekly via dual-energy X-ray absorptiometry (DXA) scans. After 4 weeks of treatment animals were sacrificed and analyzed by microCT, histology and immunohistochemistry (IHC).

PEGylation of NELL-1 increased systemic half-life from 5.5 hrs up to 31.3 hrs, with no significant detrimental effect on in vitro bioactivity. When investigating PEG-NELL-1's in vivo potential, DXA scans revealed a gradual and significant improvement in femoral BMD with PEG-NELL-1 treatment (15.8% increase compared to pre-treatment values). Moreover, IHC revealed that NELL-1 treatment both upregulates osteoblast number, thus increasing bone formation, while inhibiting osteoclast activity.

In summary, intravenous PEG-NELL-1 administration significantly improves overall bone density, with a clinically reasonable dosing frequency. With a seamless translation into an osteoporotic model and no observed toxicological effect, PEG-NELL-1 possesses great promise as a future therapeutic to combat osteoporosis in both civilian and military populations.

### Example 6. Studies on Bone Repair by PEGylated NELL-1

### 1. Introduction

NEL-like molecule-1 (NELL-1) is a potent pro-osteogenic cytokine that has been demonstrated to enhance bone formation when applied locally. PEGylation is a biocompatible process in which polyethylene glycol (PEG) is attached to a protein to prolong its half-life. The primary objective of this study is to investigate the effects of systemic administration of PEG-NELL-1 on fracture repair in an open fracture model in the mouse radius; the secondary objective is to investigate effects on bone mineral density in uninjured bones.

### 2. Methods

A total of twelve CD-1 mice aged 10 weeks were subjected to 0.15 mm transverse open osteotomies of the bilateral radii. They were treated with weekly tail vein injections of PEG-NELL-1 (n=5) or PEG phosphate buffered saline (PBS) (n=7). Animals were sacrificed at week 4. Fracture healing was evaluated by micro-CT and microPET. For the microPET, F-18, is substituted for hydroxyl groups and binds to new bone; therefore uptake is higher in newly formed bone. Bone density was evaluated by Dual-energy X-ray absorptiometry (DXA) and performed on humeri and femurs.

Statistical analyses were performed using a Student's t test. A p value of <0.05 was considered significant.

### 3. Results

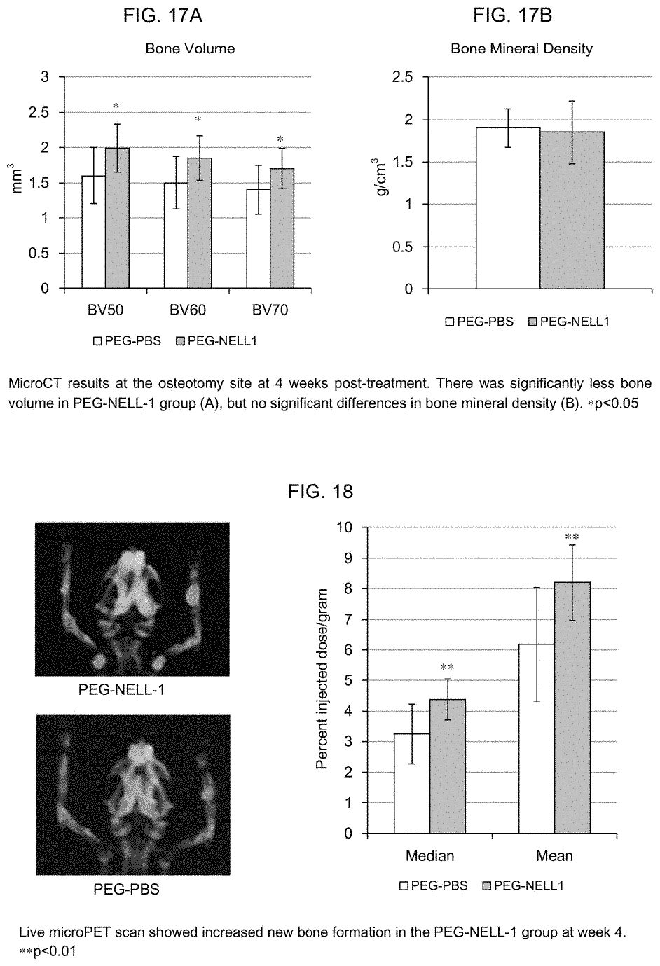

At 4 weeks of treatment, bone volume of the fracture site in the PEG-NELL-1 group was 22% greater compared with control across three different threshold values. There were no significant differences in trabecular density (FIGS. 17A-B). MicroPET demonstrated 34% more uptake of F-18 in the PEG-NELL-1 group compared with control (FIG. 18).

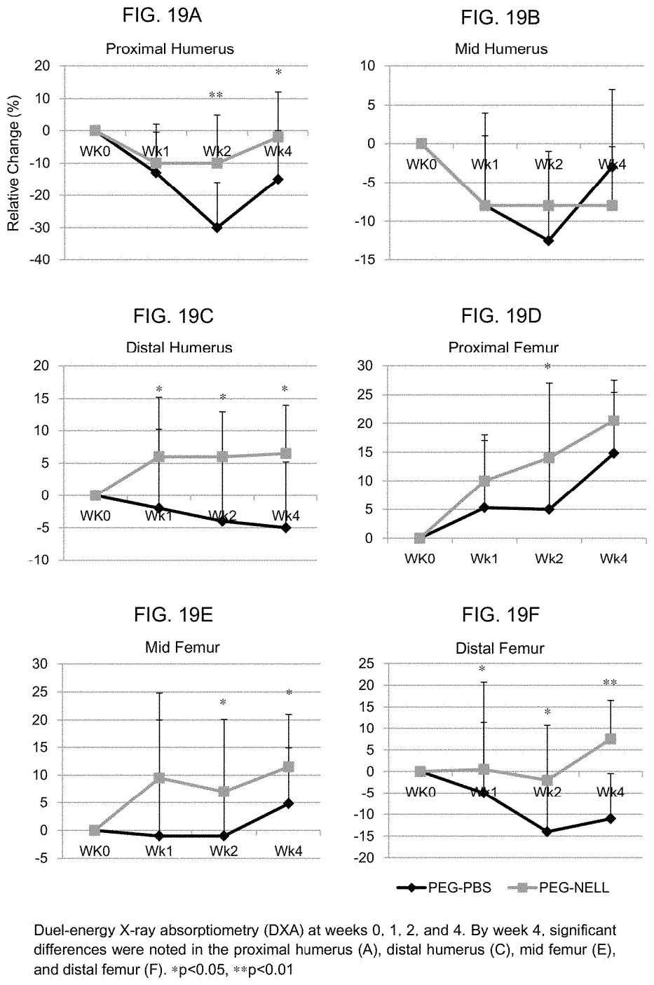

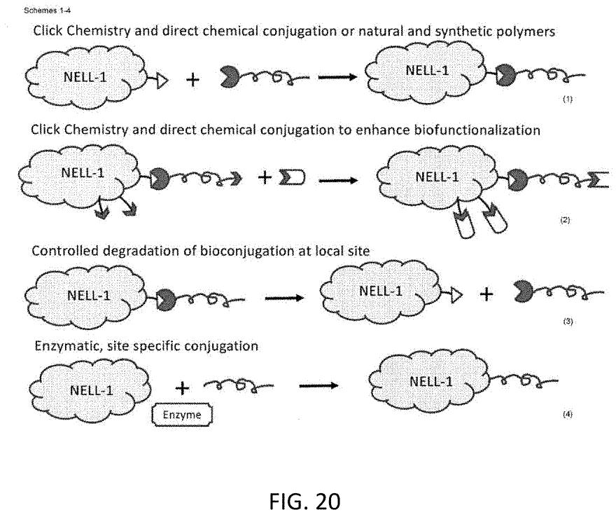

On DXA, the PEG-NELL-1 group demonstrated significantly higher BMD than control in the mid femur, distal femur, proximal humerus, and distal humerus (FIGS. 19A-F). There was no difference in BMD in the mid humerus.

### 4. Conclusion

Weekly systemic administration of PEG-NELL-1 increased bone formation in this radial defect fracture model as verified by bone volume on micro-CT and F18 uptake on microPET.

Furthermore, systemically injected PEG-NELL-1 induced bone formation as measured by DXA in uninjured long bones in mice. These exciting results warrant further investigation of PEG-NELL-1 as an injectable therapeutic agent to induce bone formation in both fractured and uninjured bones. This discovery is novel as the first demonstration of a systemically administered anabolic cytokine to enhance bone formation in fractured and uninjured long bones.

Those skilled in the art will know, or be able to ascertain, using no more than routine experimentation, many equivalents to the specific embodiments of the invention described herein. These and all other equivalents are intended to be encompassed by the following claims.

We claim:

1. A random conjugate molecule (CMN), comprising a formula of CG-X-N,
wherein:
CG is at least one chemical group,
X is a linking group, and
N is NELL-1 protein or peptide.

2. The CMN of claim 1, wherein the at least one chemical group is an alkyl group, an aryl group, an acyl group, a leaving group, a polymer, or a peptide, or a combination thereof.

3. The CMN of claim 1, wherein the chemical group is poly(ethylene glycol) (PEG), poly(ethylene oxide) (PEO), poly(propylene glycol) (PPG), or poly(propylene oxide) (PPO).

4. The CMN of claim 1, wherein the chemical group is selected from heparin sulfate, glycopolymers, zwitterionic polymers, hyperbranched polymers, polymers containing unnatural amino acids, linkers to lysine or cysteine, or peptide sequences that modify NN or CMN interactions with ECM, target cells, immune cells, and hepatocytes.

5. The CMN of claim 1, wherein the linking group comprises a responsive linker that degrades on demand to external stimuli.

6. The CMN of claim 1, wherein the linking group provides a linking reaction that is azide-alkyne, azide-BMCO, or Tetrazine-TCO type reactions.

7. The CMN of claim 1, wherein the linking group comprises a linker to lysine or cysteine.

8. The CMN of claim 1, wherein the linking group comprises a natural enzyme.

9. The CMN of claim 1, wherein the at least one chemical group imparts at least one desirable property to the NELL-1 protein such that the CMN is significantly improved in the at least one desirable property relative to a naked NELL-1 protein without chemical modification (NN).

10. A composition, comprising a random conjugate molecule (CMN) according to claim 1.

11. The composition of claim 10, further comprises a pharmaceutically acceptable carrier.

12. The composition of claim 11, which is a formulation for systemic or local delivery.

13. A method of preparing a random conjugate molecule (CMN) of a formula of CG-X-N, comprising:
a) providing a NELL-1 protein or peptide (N),
b) providing a chemical compound comprising a chemical group (CG),
c) providing a linking group that comprises a linker (X), and
d) bringing the CG into contact with the NELL-1 protein or peptide to cause conjugation between the chemical group and the NELL-1 protein or peptide to occur to form the CMN,
wherein CG, X, and N are as defined according to claim 1.

14. A method of forming a composition, comprising
providing an amount of a random conjugate molecule (CMN) of a formula of CG-X-N, and
forming the composition,
wherein CG, X, and N are as defined according to claim 1.

15. The method of claim 14, wherein the composition further comprises a pharmaceutically acceptable carrier.

16. A method of treating or ameliorating a condition in a subject, comprising administering to the subject a random conjugate molecule (CMN) of a formula of CG-X-N,
wherein CG, X, and N are as defined according to claim 1.

17. The method of claim 16, wherein the CMN is included in a composition that comprises the CMN and a pharmaceutically acceptable carrier.

18. The method of claim 16, wherein the bone condition is osteoporosis.

19. The method of claim 17, wherein the bone condition is osteoporosis.

20. The method of claim 16, wherein the bone condition is bone fracture or intervertebral disc disease or injury.

21. The method of claim 16, wherein administering comprises local or systemic administration.

22. The method of claim 16, wherein the subject is a human being.
